(19) United States
(12) Patent Application Publication
(10) Pub. No.: US 2024/0412277 A1
(43) Pub. Date: Dec. 12, 2024

(54) APPARATUS, SYSTEMS AND METHODS FOR CREATING, ACTIVATING, DISPLAYING IN A COMPUTER DISPLAY, AND INTERACTING WITH, A VIRTUAL THREE-DIMENSIONAL DIGITAL ANALOG TO TRADITIONAL PHYSICAL SHOPPING IN A PHYSICAL MARKETPLACE

(71) Applicant: Marilyn R Khorsandi, Laguna Hills, CA (US)

(72) Inventor: Marilyn R Khorsandi, Laguna Hills, CA (US)

(21) Appl. No.: 18/734,921

(22) Filed: Jun. 5, 2024

Related U.S. Application Data
(60) Provisional application No. 63/471,064, filed on Jun. 5, 2023.
(60) Provisional application No. 63/471,813, filed on Jun. 8, 2023.

(51) Int. Cl. G06Q 30/0601; A61L 9/12; A63F 13/65; A63F 13/792; G06Q 30/0251

(52) U.S. Cl. CPC G06Q 30/0643; A61L 9/125; A63F 13/65; A63F 13/792; G06Q 30/0259; A61L 2209/11; A63F 2300/8082

(57) ABSTRACT

Disclosed herein are exemplary apparatus, systems and methods for creating, displaying in a computer display, activating and interacting with a shared, interactive shopping session between at least one computer user and at least one persistent, visually traversable, interactive, persistent, virtual three-dimensional space, wherein said virtual three-dimensional space may comprise a virtual three-dimensional store, a virtual three-dimensional mall or other virtual three-dimensional shopping space. Further disclosed herein is an automatic foreign language translator that would cross-translate cross-communications between a plurality of virtual shoppers virtually shopping at a particular exemplary virtual shopping space at a particular time. Further disclosed herein is an exemplary fragrance emitting device that would receive instructions from a user's computer device, that would receive fragrance emitting instructions generated by an exemplary virtual shopping space embodiment of the present invention according to a virtual location of an exemplary online shopping user in the virtual shopping space.

The present application further claims priority to, and herein incorporates by reference in full, as if fully stated herein, the provisional U.S. patent application No. 63/471,813, entitled “APPARATUS, SYSTEMS AND METHODS FOR DYNAMICALLY CREATING, ACTIVATING, AND DISPLAYING IN A TWO-DIMENSIONAL COMPUTER DISPLAY, DYNAMIC INTERACTIVE ADVERTISING FOR INTERACTING WITH A SHARED, INTERACTIVE SHOPPING SESSION IN A PERSISTENT, VISUALLY TRAVERSABLE, VISUALLY ROTATABLE, INTERACTIVE, VIRTUAL THREE-DIMENSIONAL MARKETPLACE” (sometimes referred to herein as the “DYNAMIC INTERACTIVE ADVERTISING APPLICATION”) the full contents and disclosures of which are incorporated herein by reference as if fully stated herein.

### SUMMARY OF THE INVENTION

Exemplary embodiments of the apparatus, systems and methods disclosed herein would enable online shoppers to “walk” among enticing displays of virtual, interactive, three-dimensional merchandise, to browse and inspect the various virtual items on display, to purchase items of interest, and to “mingle” with other online shoppers in a Virtual Reality Analog to traditional shopping in a brick-and-mortar marketplace.

Exemplary embodiments of the apparatus, systems and methods disclosed herein would provide Online Retailers with a Virtual Reality Marketplace integrated with an eCommerce Engine with which to showcase their artfully curated collections of merchandise as they have been doing in their brick-and-mortar stores.

Exemplary embodiments of the apparatus, systems and methods disclosed herein would bring online shoppers the hustle and bustle sounds of a lively shopping mall, the inviting fragrances of coffee shops and food courts, and the “community” of shopping with fellow shoppers.

Some exemplary embodiments of the apparatus, systems and methods disclosed herein would be implemented as a massively-shared, online, persistent, interactive, visually traversable, visually rotatable, virtual three-dimensional marketplace (such as, for example, a massively multi-player online game (MMOG)) integrated with an electronic commerce engine.

The VIRTUAL STORE APPLICATION and the exemplary apparatus, systems and methods disclosed herein would enable creating, displaying in a computer display, activating and interacting with a shared, interactive shopping session between at least one computer user and at least one persistent, visually traversable, interactive, virtual three-dimensional store.

The VIRTUAL STORE APPLICATION and the exemplary apparatus, systems and methods disclosed herein would enable creating, displaying in a computer display, activating and interacting with a shared, interactive shopping session between a computer user and a persistent, visually traversable, interactive, virtual three-dimensional representation of a store, comprising creating and displaying in a computer display, a persistent, visually traversable, visually rotatable, interactive, virtual three-dimensional representation of a plurality of items of merchandise for sale within said persistent, visually traversable, interactive, virtual three-dimensional representation of said store.

The VIRTUAL STORE APPLICATION and the exemplary apparatus, systems and methods disclosed herein would further enable creating and displaying in a computer display, a Metaverse/Virtual Reality shopping session between a computer user and a virtual store, comprising creating and displaying in a display, a persistent, visually traversable, visually rotatable, interactive, virtual three-dimensional representation of a plurality of virtual items of merchandise virtually positioned on visually traversable, virtual three-dimensional representations of a plurality of display structures that are virtually positioned within an interactive virtual store.

In some illustrative exemplary embodiments of the invention disclosed in the VIRTUAL STORE APPLICATION, and the exemplary apparatus, systems and methods disclosed herein would further provide for the creation of a virtual shopping center or marketplace (which may sometimes herein be referred to as a “mall embodiment,” “virtual mall embodiment,” or “virtual marketplace embodiment”) that would in turn comprise a plurality of persistent, visually traversable, interactive, virtual three-dimensional representations of virtual stores, and that would in turn also provide for the creation of one or more persistent, visually traversable, interactive, virtual three-dimensional representations of common area features.

The VIRTUAL STORE APPLICATION, and the exemplary apparatus, systems and methods disclosed herein would further provide for an exemplary fragrance emitting device that would be communicatably connected with a user's computer device, that would receive fragrance emitting instructions originated by an exemplary mall or store embodiment of the present invention, and that would emit the particular fragrance according to the received instructions.

The VIRTUAL STORE APPLICATION, and the exemplary apparatus, systems and methods disclosed herein would further provide for instructing an exemplary fragrance emitting device to emit a particular fragrance of a plurality of fragrances at a particular intensity for a particular duration of time.

The VIRTUAL STORE APPLICATION, and the exemplary apparatus, systems and methods disclosed herein would further provide for an automatic foreign language translator that would cross-translate cross-communications between a plurality of virtual shoppers virtually shopping at a particular exemplary virtual store or mall.

The present application further discloses exemplary apparatus, systems and methods for dynamically creating, activating, and displaying in a computer display, dynamic interactive advertising for interacting with a shared, interactive shopping session in a persistent, visually traversable, visually rotatable, interactive, virtual three-dimensional marketplace.

Online retailers have been presenting online shoppers with online shopping experiences that respond to a user's search for a particular type of item with a two-dimensional list of pictures and descriptions of items that might match the user's search. Artificial Intelligence is being tapped to provide more sophisticated responses to a user's online shopping search. But the result has nevertheless been in the form of a two-dimensional list of pictures of items of merchandise with corresponding information about each item.

As compared to such two-dimensional Search-Responsive Lists of products, exemplary embodiments described herein would provide shopping users with a digital, virtual reality, virtual three-dimensional analog to a traditional, three-dimensional physical shopping experience in a physical marketplace. In particular, the digital, virtual reality, virtual three-dimensional shopping marketplace that would be provided by exemplary embodiments of the present invention would allow an online shopping user to “walk” (sometimes referred to herein as “visually traversing”) through a mall or store. The online shopping user would be able to browse store fronts, or if virtually “in” a virtual store, browse various virtual items of merchandise in various virtual departments in virtual three-dimensional displays that have been curated by virtual store owners.

The online shopping user would be able to virtually examine (sometimes referred to herein as visually rotate) various virtual items of merchandise before deciding whether or not to purchase the item(s).

As part of providing a shopping user with a close analog to physical shopping, the digital, virtual reality, virtual three-dimensional shopping marketplace that would be provided by exemplary embodiments of the present invention would continuously track the location of each shopping user that is shopping in the virtual shopping marketplace at a given time, and would, with the authorization of each shopping user, display each shopping user's presence, such as in the form of a Shopping Emoji (or Shopping Avatar, or Player GameObject, or the like, as the case may be) to each of the other online shopping users that are shopping in the virtual shopping marketplace at that given time; sounds (like the hustle and bustle of a busy store or mall) and even smells (for example fresh coffee brewing from a Coffee Shop or food smells from a virtual Food Court) in some exemplary embodiments would be generated.

Various exemplary embodiments disclosed in the present application sometimes describe displaying and interacting with a virtual three-dimensional environment in a two-dimensional computer display. As will be understood by someone with ordinary skill in the art, virtual reality headsets are currently available. Exemplary embodiments of the present invention could be implemented using virtual reality headsets as what is referred to herein as a shopping user computer device, a playing user computer device, a player computer device, or the like. As will be understood by someone with ordinary skill in the art, some exemplary embodiments would display an exemplary virtual shopping session to, and interact with, a virtual reality headset without departing from the spirit of the present invention. Some exemplary embodiments would display to, and interact with, various types of shopping user computer devices and various types of computer display devices, including those that use two-dimensional display devices, and those that comprise or interact with or interface with virtual reality headsets, or with other computer devices, whether now known or in the future discovered, without departing from the spirit of the present invention.

In some exemplary embodiments, an exemplary shared, interactive shopping session would be provided as part of an exemplary Massively Multi-Player Online Shopping Game (sometimes referred to herein as an exemplary “Shopping MMOG” or as an exemplary “MMOSG”). In some such exemplary MMOSG embodiments, a game engine, such as, by way of non-limiting example, Unity 3D, would be used to create an exemplary “Front End” that would be interactive with a plurality of Players; the exemplary “Front-End” would comprise a plurality of virtual three-dimensional (“3D”) game objects (“GameObjects”); a scripting language such as, by way of non-limiting example, C #, would be used to program the behavior of each virtual 3D GameObject in response to interaction with other virtual 3D GameObjects. Some such exemplary MMOSG embodiments would present a plurality of virtual three-dimensional (“3D”) game objects (“GameObjects”) representing items of merchandise (sometimes referred to herein as “virtual 3D merchandise items”); each virtual 3D merchandise item would be virtually selectable, such as for examination and possible purchase, by virtual 3D Player GameObjects; each virtual 3D Player GameObject would represent a particular virtual “Shopper;” each virtual 3D Player GameObject would be programmed to interactively function, and move, according to instructions from a particular shopping user.

### BRIEF DESCRIPTION OF DRAWINGS

FIG. 1A illustratively, graphically depicts a front, two-dimensional view of an exemplary virtual box in an exemplary embodiment of the present invention;

FIG. 1B illustratively, graphically depicts a virtual, three-dimensional view of an exemplary virtual box in an exemplary embodiment of the present invention;

FIG. 2A illustratively, graphically depicts a virtual, three-dimensional view of an exemplary virtual box before it has been proportionally scaled in an exemplary embodiment of the present invention;

FIG. 2B illustratively, graphically depicts a virtual, three-dimensional view of a proportionally scaled virtual box in an exemplary embodiment of the present invention;

FIG. 2C illustratively graphically depicts an illustrative, exemplary proportional scaling of a three-dimensional teddy bear to a proportionally scaled virtual teddy bear on a virtual shelving unit in an exemplary virtual store in an exemplary embodiment of the present invention;

FIG. 3 illustratively depicts an exemplary high-level logic flow diagram for proportionally scaling an exemplary item of merchandise to fit on a proportionally scaled virtual exemplary display structure in an exemplary embodiment of the present invention;

FIG. 4 illustratively, graphically depicts two virtual shopping users in an exemplary shared virtual shopping session in an exemplary virtual store in an exemplary embodiment of the present invention;

FIG. 5 illustratively, graphically depicts an exemplary fragrance emitting device communicatably connected with an exemplary shopping user's computer device in an exemplary embodiment of the present invention;

FIG. 6 illustratively depicts an exemplary high-level logic flow diagram of an exemplary embodiment of the present invention instructing an exemplary fragrance emitting device to emit a particular fragrance at a particular intensity for a particular duration;

FIG. 7 illustratively depicts an exemplary high-level logic flow diagram of an exemplary mall embodiment of the present invention determining requirements for sending fragrance instructions for a particular fragrance to a particular virtual shopping user;

FIG. 8 illustratively depicts an exemplary diagram of components for an exemplary fragrance emitting device in an exemplary embodiment of the present invention;

FIG. 9 illustratively depicts an exemplary closeup view of an exemplary fragrance emitting element in an exemplary fragrance emitting device in an exemplary embodiment of the present invention;

FIGS. 10A-10B illustratively present a high-level logic flow diagram depicting an exemplary advertising logic flow in an exemplary embodiment of the present invention;

FIG. 11 illustratively depicts an exemplary Shopping User/Shopping Player as an exemplary First-Person-Shopper (“FPS”) entering an exemplary virtual Mall in an exemplary MMOSG mall embodiment;

FIG. 12 illustratively depicts an exemplary Shopping User's/Shopping Player's Shopping Emoji in an exemplary Mall in front of an exemplary virtual Store Front; shopping mall sounds would be heard; the Shopping Emoji grabs a mall Shopping Cart;

FIG. 13 illustratively depicts an exemplary Shopping User's/Shopping Player's Shopping Emoji shopping in an exemplary virtual Store; exemplary Store sounds would be heard; other virtual Shopping Users'/Shopping Players' Shopping Emojis that are shopping in the same virtual area of the virtual exemplary Store would be visible to each other; each exemplary Shopping User/Shopping Player can hear the sound effects of, and see, the exemplary placement of an exemplary virtual three-dimensional item of merchandise in the Shopping Emoji's virtual cart;

FIG. 14 illustratively depicts an exemplary Shopping User's/Shopping Player's Shopping Emoji viewing a plurality of exemplary virtual three-dimensional items of merchandise displayed on an exemplary virtual table;

FIG. 15 illustratively depicts the exemplary Shopping User's/Shopping Player's Shopping Emoji touching one of the plurality of exemplary virtual three-dimensional items of merchandise displayed on the exemplary virtual table; touching the exemplary item and holding the touch is illustratively depicted to cause an exemplary interactive display of information about the item, such as, by way of non-limiting, illustrative example, among other things: price, manufacturer, material, where made, and the like;

FIG. 16 illustratively, graphically depicts an exemplary shared Online Shopping Marketplace System illustratively interacting with an exemplary eCommerce Engine, which in turn accesses a number of various databases of information relevant to the Online Shopping Marketplace;

FIG. 17 illustratively depicts the exemplary Shopping User's/Shopping Player's Shopping Emoji leaving the exemplary virtual table displaying the plurality of exemplary virtual three-dimensional items of merchandise;

FIG. 18 illustratively depicts an exemplary Shopping User's/Shopping Player's Shopping Emoji approaching an exemplary virtual table displaying a plurality of exemplary virtual three-dimensional items of merchandise;

FIG. 19 illustratively depicts the exemplary Shopping User's/Shopping Player's Shopping Emoji examining a particular exemplary virtual three-dimensional item of merchandise that the exemplary Shopping Emoji picked up from the exemplary virtual table; and

FIG. 20 illustratively depicts the exemplary Shopping User's/Shopping Player's Shopping Emoji placing the particular exemplary virtual three-dimensional item of merchandise in the Shopping Emoji's virtual cart; sound effects are illustratively graphically depicted as being heard from the exemplary placement of the exemplary virtual three-dimensional item of merchandise in the Shopping Emoji's virtual cart.

### DEFINITIONS

The phrase “virtual three-dimensional” as used herein refers to graphically depicting a three-dimensional physical object in digital form so that the graphic depiction of the three-dimensional (“3D”) object provides the appearance of the digital form having three-dimensions—this means that even in a two-dimensional display, such as a two-dimensional computer display monitor, special graphic treatments of the display of the object cause the two-dimensional view to appear as though the object is three-dimensional. For example, an exemplary box 10a is depicted in FIG. 1a in a simple, two-dimensional, frontal view. However, by displaying the same exemplary box 10b from an angle, thus showing front, top and sides, the two-dimensional box object takes on three-dimensional features. Various other graphic features, such as, by way of non-limiting, illustrative examples, shading, lighting, subtle shadows, and the like, can further enhance three-dimensional appearance characteristics of an object displayed in a two-dimensional display. Of course, even though a graphic display that causes a two-dimensional view to appear as though the object is three-dimensional, the display is not actually a three-dimensional physical object—that is because a three-dimensional physical object actually has a plurality of sides and views; it has weight and mass. Therefore, even when the object is displayed in a two-dimensional display with graphic 3D effectw, because the display merely takes on graphic three-dimensional characteristics, the phrase “virtual” three-dimensional is used.

Similarly, the phrase “virtual three-dimensional” also refers to a digital representation of a three-dimensional physical object for viewing with virtual reality devices. That is because, as realistic as such devices may cause virtual 3D objects to appear, virtual 3D objects do not have weight and mass and are therefore not physical.

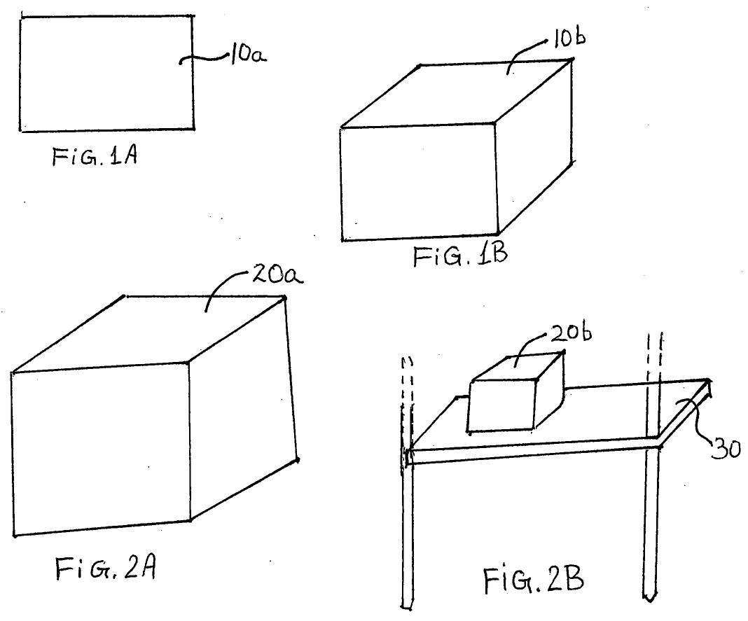

The phrase “proportionally scale” as used herein refers to modifying the size of a graphic display of a virtual three-dimensional object to graphically “fit” in a computer display, (such as, for example, a two-dimensional computer display monitor, or a virtual reality display), and to also graphically “fit” in the graphic display of other virtual three-dimensional objects, while maintaining (i.e., without destroying) the proportional relationships between the various features of the object. For example, an exemplary virtual three-dimensional box 20a is depicted in FIG. 2A. The exemplary box 20a is large, and if depicted in this size, would block the display of other portions of a computer display. In order to graphically “fit” the exemplary virtual three-dimensional box 20a in a computer display, every aspect of the size of box 20a when proportionally sized, would result in exemplary virtual three-dimensional box 20b depicted in FIG. 2B, which has been scaled to “fit” not only a computer display, but to also graphically “fit” on exemplary virtual three-dimensional display structure 30 (an exemplary shelving unit). Herein, the exemplary miniaturization of exemplary box 20a into exemplary box 20b is referred to as proportionally scaling. However, proportional scaling would not be limited to size reduction (i.e., miniaturization), but may also refer to proportionally enlarging a graphic depiction of an item.

FIG. 2C illustratively graphically depicts an illustrative, exemplary proportional scaling of a three-dimensional teddy bear 40a to a proportionally scaled virtual teddy bear 40b on a virtual shelving unit 50 in a virtual store 60; exemplary dashed lines 55a and 55b graphically depict the ratio of proportional scaling from the original size of the three-dimensional teddy bear 40a to the proportionally scaled virtual teddy bear 40b.

The phrase “visually traversable” as used herein refers to a graphic progression, such as, by way of non-limiting, illustrative examples: through a store, or along an isle in a store, or through an open area in a shopping center, or passing a display structure, such as a rack of clothing, or a shelfing unit.

The phrase “visually rotatable” as used herein refers to graphically, interactively, rotating the display view of a virtual three-dimensional object, such as by grabbing an object with a user mouse click and dragging/rotating it in a computer display.

As will be understood by someone with ordinary skill in the art, mention herein of use of a computer mouse to click on something, or to manipulate something, such as dragging or rotating it, is illustrative and non-limiting. In other exemplary embodiments, other computer keys, other manipulation devices, and/or verbal commands, or touch or look commands (such as with virtual reality headsets) whether now used or known, or in the future discovered, could be used to manipulate virtual items of merchandise or perform other exemplary interactions with an exemplary embodiment without departing from the spirit of the present invention.

### DETAILED DESCRIPTION

In one exemplary mall embodiment, a first entity (sometimes referred to herein as an exemplary “mall owner”) would contract for purchase (or lease in some exemplary mall embodiments) of virtual three-dimensional mall space (sometimes referred to herein as virtual 3D mall “real estate”). In such an exemplary mall embodiment, the exemplary mall owner would provide the exemplary embodiment of the exemplary apparatus, system or method, with exemplary requirements and/or specifications for a virtual buildout of a virtual mall and of virtual stores, including virtual mall common areas within the exemplary virtual mall real estate. In such an exemplary mall embodiment, exemplary common areas would comprise, according to a mall owner's specifications, as non-limiting examples, a water feature, a skating rink, a playground, a conversation area with benches, virtual simulation games, or a performance stage.

In such an exemplary mall embodiment, the exemplary mall owner would be charged an exemplary amount (for either a “purchase,” or lease, as agreed upon by the mall owner and the apparatus/system/method administrator (the “administrator”)), wherein said exemplary amount would comprise amounts for, by way of non-limiting examples: digital storage space, interactive data capacity, interactivity volume, interactivity speed, and virtual three-dimensional build-out requirements (for such things as virtual store buildouts, virtual special common area features, virtual interior mall design, and other virtual physical features of the exemplary virtual mall, such as virtual advertising display structures, extent of interactivity, and points of interactivity). Said exemplary amount may comprise an exemplary upfront amount, and may further comprise exemplary periodic payment amounts; periodic payment amounts could include percentages based on profits or revenues.

Once the exemplary virtual mall had been “built,” the exemplary mall owner would then contract with various exemplary store owners for “leasing” exemplary virtual stores in the exemplary mall. Each exemplary store owner would likewise need to provide virtual buildout requirements for the exemplary store owner's particular virtual store. In some exemplary mall environments, the administrator would be responsible for implementing said virtual store buildout requirements; in other exemplary mall environments, the mall owner would be responsible for implementing said virtual store buildout requirements.

In order for an exemplary store owner to “build” a virtual store, the exemplary store owner would need to provide, such as to either the mall owner or the administrator, information about, by way of non-limiting, illustrative examples: Store layout specifications; Department specifications; Isle specifications; Shelving specifications; Lighting specifications; Platform specifications; Cabinet specifications; Rack specifications; Display case specifications; Mannequin specifications; Décor specifications; Merchandise specifications; Storefront Design Specifications, including virtual storefront display window specifications (which could be persistent (subject to change by a store owner) or dynamic (to display ads pertinent to particular approaching shoppers based on shopper-specific shopping history) and Merchandise specifications.

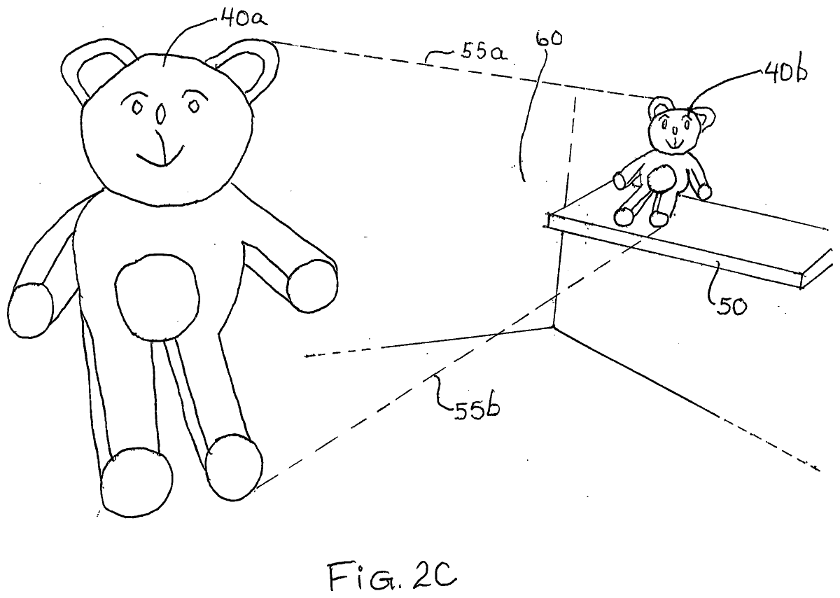

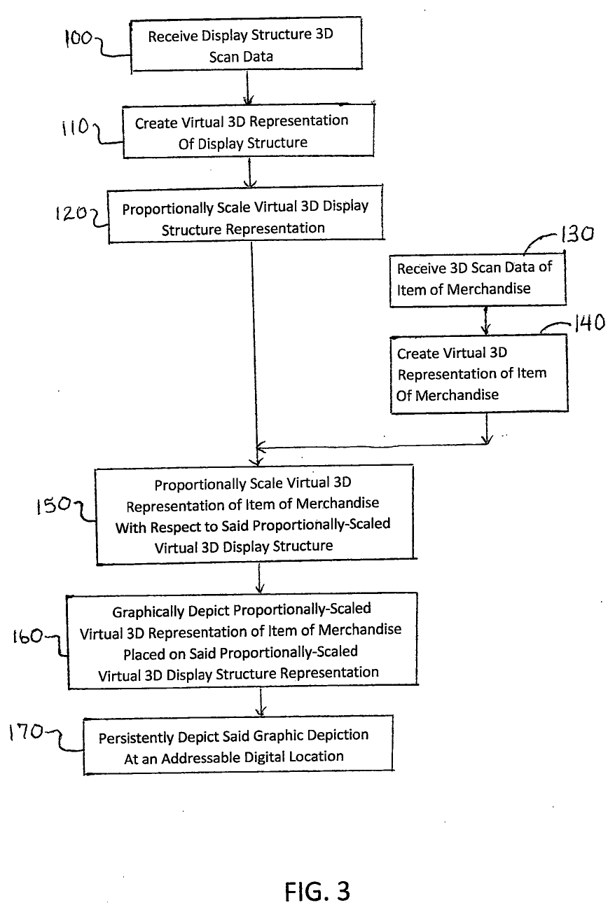

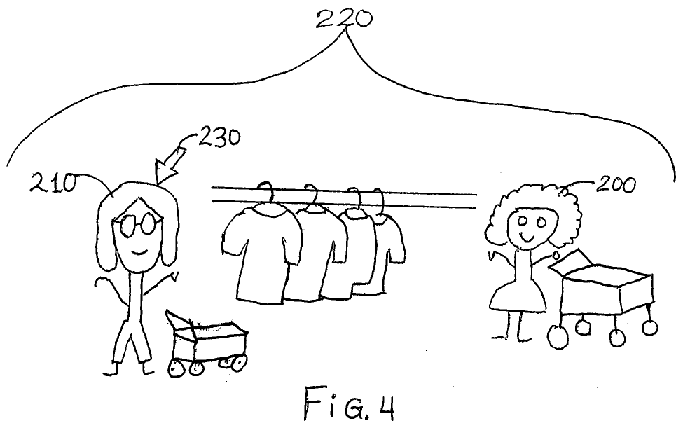

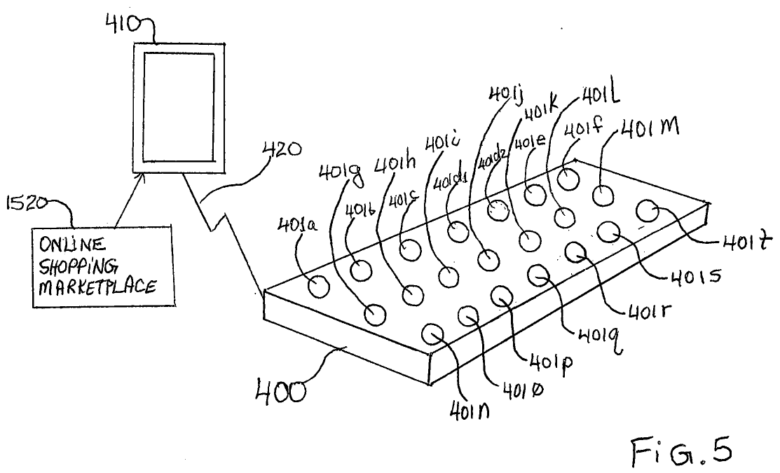

In order for an exemplary store owner to “stock” a virtual store with virtual items of merchandise, the exemplary store owner would need to provide, e.g., to the administrator, the mall owner, or in some embodiments, to a store-owner version of software for “stocking” the store, detailed information about the exemplary store owner's display structures (such as racks, shelving, and the like) and also detailed information about the items of merchandise to be stocked/displayed. An exemplary high-level logic flow of an exemplary “stocking” process is depicted in FIG. 3. As illustratively depicted in FIG. 3, an exemplary stocking aspect would comprise, for example, software, to receive 100 Display Structure three-dimensional scan data.

As will be understood by someone with ordinary skill in the art, discussion herein to receiving display structure 3D scan data is illustrative and exemplary. Without departing from the spirit of the present invention, as an alternative to scanning 3D data of an actual physical display structure, it would be possible to virtually construct exemplary virtual display structures as, e.g., a type of GameObject; in a case of a virtual rack that would support the hanging of virtual hangers that would in turn support presentation of clothing items, the virtual rack could be a first GameObject, and each virtual hanger would be separate GameObjects.

As will be understood by someone with ordinary skill in the art, three-dimensional scan data can be provided in different forms. Exemplary embodiments described herein would not be limited to any particular form of three-dimensional scan data. Rather, exemplary embodiments would be provided that could be adapted to any form of three-dimensional scan data, whether now known or in the future discovered. Whether a particular form of three-dimensional scan data provided an interior-out view of a three-dimensional object (e.g., a dense cloud of referential points representing the mass of an object), or an exterior-out view, e.g, referential points from all the surfaces of a three-dimensional object out to an encompassing referential structure's perimeters, exemplary embodiments would be provided that would be programmed to accommodate such scan data.

Reference herein to programming may include programming in any of various programming languages, whether now known or in the future discovered, including, as illustrative, non-limiting examples, C #, C++, JavaScript, Python, Rust, Solidity, and Unity, among others.

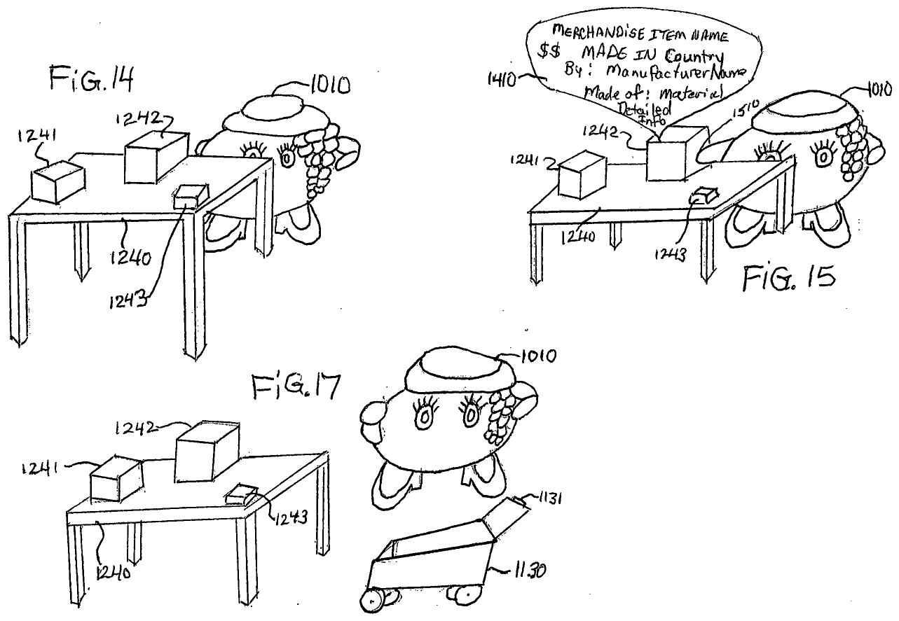

As further illustratively depicted in FIG. 3, an exemplary stocking aspect of the present invention would further comprise, for example, software, to: create 110 a virtual three-dimensional display structure from said three-dimensional scan data; proportionally scale 120 the virtual three-dimensional representation of the display structure; receive 130 three-dimensional scan data of an item of merchandise; create 140 a virtual three-dimensional representation of the item of merchandise; proportionally scale 150 the virtual three-dimensional representation of the item of merchandise with respect to the proportionally scaled virtual three-dimensional display structure representation; graphically depict 160 the virtual proportionally-scaled three-dimensional representation of the item of merchandise placed on the virtual proportionally-scaled three-dimensional representation of the display structure; persistently depict 170 the aforementioned graphic depiction at an addressable digital location, such as, for example, at a particular website. Some exemplary embodiments would store information regarding the virtual display structure and any corresponding physical display structure in an exemplary Support Structure Database such as the exemplary Support Structure Database 1570 illustratively depicted in FIG. 16.

In alternative exemplary embodiments, virtual items of merchandise would be arranged on virtual display structures, and then the entire stocked virtual display structure would be proportionally scaled for persistent depiction in the virtual store.

In the exemplary embodiment, each virtual display structure, proportionally scaled and graphically depicted as described above, would be visually traversable. That is, a virtual shopping user visiting the exemplary virtual store would be able to move from a first point near the display structure, to another point near the display structure.

In the exemplary embodiment, each display structure, proportionally scaled and graphically depicted as described above, would be “persistently” depicted. That is, whenever a shopping user visits the exemplary virtual store, and moves to a location within the virtual store where the particular display structure is virtually located (as defined by the store owner's store layout specifications), the proportionally-scaled display structure would be graphically depicted. Exemplary embodiment would provide for store owners to rearrange and/or replace virtual display structures within their virtual store.

In the exemplary embodiment, each virtual item of merchandise, proportionally scaled and graphically depicted as described above, would be visually rotatable. That is, a user would be able to select (grab) the item (e.g., by clicking the item and dragging it) and would be able to rotate the graphic depiction, similar to a shopper being able to handle an actual three-dimensional item.

In the exemplary embodiment, a plurality of virtual items of merchandise could be “stocked” on a single virtual display structure. For example, a plurality of clothing items could be hung on virtual hangers hanging on a single virtual display rack. Similarly, a plurality of small appliances could be virtually place on a single virtual display shelf.

In the exemplary embodiment, the exemplary stocking aspect would be driven by artificial intelligence programming that has been trained to receive display requirements for creating proportionally scaled arrangements of a plurality of virtual items of merchandise on virtual display structures.

In one exemplary mall embodiment, the exemplary virtual mall would be accessible at a particular addressable digital location, such as a website, on the Internet, or alternatively, a particular addressable digital location on the Metaverse.

In some exemplary embodiments, instead of a virtual location within a virtual mall, an exemplary virtual store owner would contract directly with an administrator to establish an exemplary virtual store at a particular addressable location, such as a website (sometimes referred to herein as an exemplary “website store” embodiment), on the Internet, or alternatively, a particular addressable location on the Metaverse. In an exemplary embodiment where the exemplary virtual store is located at a particular addressable location on the Metaverse, an exemplary interactive virtual storefront associated with the exemplary virtual store would, for example, be visually presented on a virtual “street” in the Metaverse (sometimes referred to herein as a “street store” embodiment).

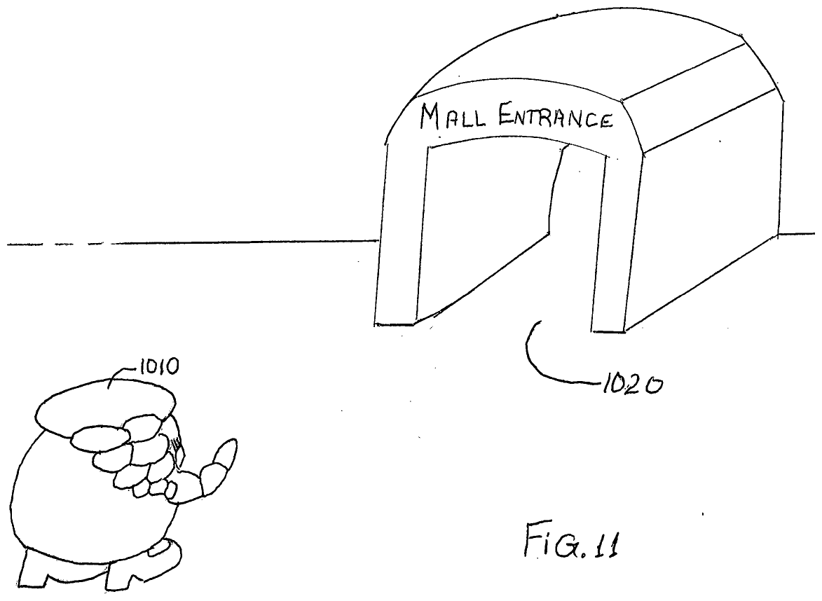

In one exemplary mall embodiment, when an exemplary shopping computer user chooses to navigate to enter a particular exemplary interactive virtual mall, the exemplary embodiment would present an exemplary virtual entry into the exemplary interactive virtual mall and an exemplary interactive view of an exemplary interior of the particular exemplary interactive virtual mall (Cf., e.g., FIG. 11 as described elsewhere herein0. From the exemplary virtual mall entry, the exemplary interactive view would engage with the exemplary shopping computer user to continuously receive user location changes, which in some exemplary embodiments would be controlled, by way of nonlimiting examples, by the exemplary shopping computer user using an exemplary computer “mouse” to move about the exemplary interactive virtual mall. For example, the exemplary shopping computer user could navigate the mouse to move and click a cursor arrow, and/or, could use an exemplary mouse scroll roller. The exemplary interactive virtual mall would show exemplary virtual storefronts. F., e.g., FIG. 12 as described elsewhere herein). When the exemplary computer user sees an exemplary virtual storefront of interest, the exemplary computer user could, for example, double click on that storefront, to enter the particular exemplary virtual store associated that with particular exemplary virtual storefront. In some exemplary mall embodiments, a virtual mall directory would be provided such as near the virtual entrance of the virtual mall—each virtual store location (and each virtual mall common area location would provide an interactive virtual selection button that, if interactively engaged by the shopping computer user, would navigate the shopping computer user to a virtual location corresponding to the engaged store or common area button.

In an exemplary street store embodiment, the exemplary computer user would interactively navigate through a Metaverse environment to find an exemplary virtual storefront for the exemplary street store. The exemplary computer user could then enter the exemplary street store by, for example, double-clicking on the exemplary virtual storefront for the exemplary street store.

As will be understood by someone with ordinary skill in the art, there are various ways to navigate an online virtual 3D space-mention herein of using an exemplary computer “mouse” to click a cursor arrow, or use an exemplary mouse scroll roller, are exemplary, illustrative and non-limiting. Rather, without departing from the spirit of the present invention, in other exemplary embodiments, other computer keys, other manipulation devices, and/or verbal commands, and/or or touch or look commands (such as with virtual reality headsets) whether now used or known, or in the future discovered, could be used to enter, or otherwise navigate through, a virtual 3D marketplace and/or store.

In an exemplary website store embodiment, the exemplary shopping computer user could use a web browser to navigate to an exemplary website store. In such an exemplary website store embodiment, an exemplary shopping computer user would virtually enter a virtual store by instructing a computer device controlled by the shopping exemplary computer user to connect to a website that has been programmed to provide a shared, visual, interactive, virtual, three-dimensional shopping experience.

Once the exemplary shopping computer user (sometimes referred to herein as the exemplary “shopping user”) is virtually “inside” an exemplary interactive virtual 3D store, an exemplary embodiment would provide an exemplary interactive view of the interior of the exemplary interactive virtual 3D store. The exemplary embodiment would engage with the exemplary shopping user to receive user location changes (in some exemplary embodiments, receiving user location would be continuous), which in some exemplary embodiments would be controlled, by way of non-limiting examples, by the exemplary computer user using an exemplary computer “mouse” to move about the exemplary interactive virtual 3D store. For example, the exemplary shopping user could navigate the mouse to move and click a cursor arrow, and/or, could use an exemplary mouse scroll roller. The exemplary interactive virtual 3D store would show exemplary virtual 3D display structures, such as, by way of non-limiting examples, exemplary virtual 3D racks, shelves, platforms, mannequins, and tables. When the exemplary shopping user sees an exemplary virtual 3D display structure of interest (as an illustrative, non-limiting example, a virtual 3D rack with virtual 3D clothing items on virtual 3D hangers hanging on the virtual 3D rack), the exemplary shopping computer user could, for example, double click on that exemplary virtual 3D display structure to traverse the interior of the exemplary virtual store to get close to the exemplary virtual 3D display structure of interest.

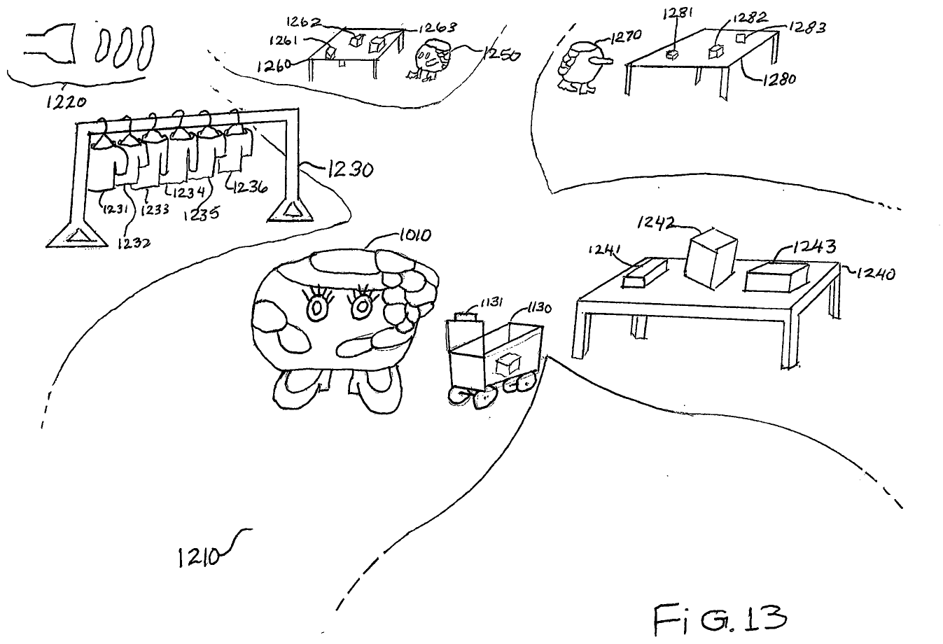

The exemplary virtual 3D display structure of interest could present a sign, such as a “Sale” sign, indicating a sale of the items on, in, or otherwise associated with, the exemplary virtual 3D display structure of interest. The exemplary virtual 3D display structure could further present pricing information, size and/or other specification information regarding virtual 3D items of merchandise associated with the exemplary virtual 3D display structure of interest. Further, pricing information, and/or size, and/or other specification information regarding a particular virtual 3D item associated with the exemplary virtual 3D display structure could be presented on the particular virtual 3D item, hovering over the particular virtual 3D item, or otherwise associated with the exemplary particular virtual 3D item. In some exemplary embodiments, presentation of such item information for a particular virtual 3D item could be triggered by an exemplary shopping user clicking once on the particular virtual 3D item. (Cf., e.g., FIG. 15 as described elsewhere herein.)

In one exemplary embodiment, virtual items of merchandise would be displayed on virtual 3D display structures; a set of specifications for each virtual 3D item of merchandise would be saved in a “back-end” “eCommerce” database; the back-end, eCommerce database would be accessible to the exemplary “front-end” shopping embodiment computer system; an exemplary shopping user would be able, for example, to click the shopping user's cursor on a particular virtual 3D item of merchandise, and that click would cause the exemplary front-end shopping embodiment computer system to access the exemplary back-end eCommerce database for exemplary specifications associated with the particular virtual 3D item of merchandise and to display, such as in an exemplary display bubble associated with the particular item of merchandise, the exemplary specifications (e.g., the item price and size, manufacturer, brand, etc.) for that exemplary virtual 3D item of merchandise. (Cf., e.g., FIG. 16 and exemplary Merchandise Item Database 1550 as described elsewhere herein.)

In the exemplary embodiment, each shopping user would have set up, such as in a back-end eCommerce database, an Account with payment details (e.g., a wallet), contact information, and any other information (including security features) needed for transacting with the exemplary embodiment. (Cf., e.g., FIG. 16 and exemplary Account Database 1580 as described elsewhere herein.)

Exemplary presentation of an exemplary virtual 3D item would present a visually rotatable, virtual three-dimensional representation of the exemplary virtual 3D item, allowing the shopping user, using, for example, a computer mouse (or other keys or manipulation devices or features, whether now known or in the future discovered), to select/grab the exemplary virtual 3D item, and virtually inspect the exemplary virtual 3D item by causing rotation of the exemplary virtual item by rolling it forward, rolling it backward, turning it clockwise, and/or turning it counterclockwise. That is, an exemplary shopping user would be able to grab a virtual item of merchandise and turn it around to view it from various angles. (Cf., e.g., FIG. 19 as described elsewhere herein.) In an exemplary case such as the aforementioned package of light bulbs, the aforementioned rotation of the exemplary package of light bulbs would provide the exemplary shopping user with a view of the back of the package, and a view of the top of the light bulbs, as well as a view of the bottom of the light bulbs.

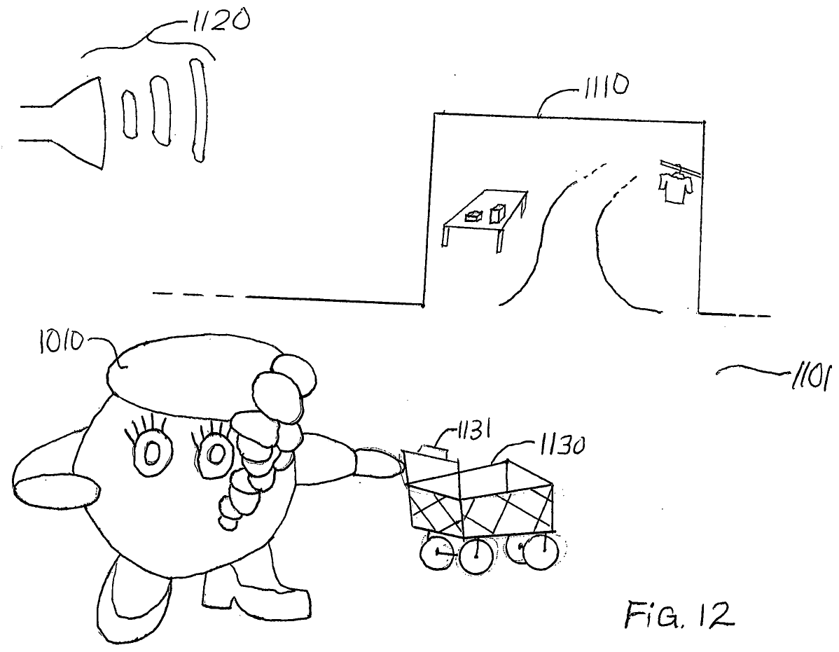

If the exemplary shopping user was satisfied with the exemplary virtual 3D item, the exemplary shopping user would be able to put the exemplary virtual 3D item in an exemplary virtual 3D shopping cart. (Cf. e.g., FIG. 20, as described elsewhere herein.) In an exemplary embodiment, an exemplary virtual 3D shopping cart would follow the virtual representation of the exemplary shopping user wherever the exemplary shopping user moves within the exemplary virtual 3D store. (Cf., e.g., exemplary virtual cart 1130 as illustratively depicted in, e.g., FIGS. 12, 13.)

In the exemplary embodiment, the exemplary shopping user could use the computer mouse to click and hold the exemplary virtual item and drag and drop it in the user's exemplary virtual 3D shopping cart. (Cf., e.g., FIG. 20 as described elsewhere herein.)

As will be understood by someone with ordinary skill in the art, mention herein of use of a computer mouse to click on something, or to manipulate something, is illustrative and non-limiting. In other exemplary embodiments, other computer keys, other manipulation devices, and/or verbal commands, whether now used or known, or in the future discovered, could be used to manipulate virtual items of merchandise or perform other exemplary interactions with an exemplary embodiment.

As will be further understood by someone with ordinary skill in the art, mention herein of storage of information in a back-end eCommerce database (such as item Specifications, shopping user Account information, and the like) is illustrative and exemplary; such information could be stored and accessed in any fashion whether now known or in the future discovered.

If, on the other hand, the exemplary shopping user was not interested in the exemplary virtual 3D item, the exemplary shopping user could use the computer mouse to click and hold the exemplary virtual item and drag and drop it back on to the exemplary virtual display structure.

In some exemplary virtual stores, exemplary virtual shopping isles would be presented, lined on each side with virtual 3D shelves, wall-hanging racks, and bins of various types. In other exemplary virtual stores, exemplary virtual 3D floor-mounted, and wall-mounted racks would be presented with exemplary virtual 3D clothing items on virtual 3D hangers hanging from the virtual 3D racks; folded virtual 3D clothing items would be presented such as on an exemplary virtual 3D table or shelf. (Cf., e.g., FIG. 13 as described elsewhere herein.)

In one exemplary virtual 3D store, exemplary virtual 3D clothing items would be folded and presented in an exemplary virtual 3D stack, such as on a virtual 3D table or on a virtual 3D shelf; an exemplary shopping user selection of an exemplary virtual 3D item from the exemplary virtual 3D stack would cause the exemplary virtual 3D item to be virtually removed from the exemplary virtual 3D stack and virtually unfolded for presentation to the exemplary shopping user for inspection. In such an exemplary embodiment, the exemplary shopping user could, for example, right-click on the unfolded exemplary virtual 3D item in order to cause a display of detailed information about the exemplary virtual 3D item, such as, by way of illustrative, non-limiting examples: retail price; sale price; size; fiber content; place of manufacture; washing instructions; consumer reviews; an option to show a video of the item, use of the item, and/or installation of the item, etc. As mentioned illustratively elsewhere herein, such detailed information about a virtual 3D item could be stored in an exemplary back-end eCommerce database and accessed at the time a shopping user indicates interest in the virtual 3D item. (Cf., e.g., FIG. 16, exemplary Merchandise Item Database 1550 accessed 1551 by exemplary eCommerce Engine 1530 in response to exemplary access 1590 by exemplary Online Shopping Marketplace 1520 in response to exemplary shopping user 1010 touching 1510 exemplary virtual Item of Merchandise 1242.) If the exemplary shopping user chooses to return the exemplary virtual item to the exemplary virtual stack, the exemplary embodiment would cause the exemplary virtual item to be virtually folded and put back in the exemplary virtual 3D stack.

Exemplary embodiments would provide an exemplary store-owner a virtual display design system that would be useable by exemplary store owners to arrange virtual displays of virtual Items of Merchandise on virtual display structures.

The exemplary store owner may display some virtual 3D items unfolded, in close proximity to a virtual stack of virtual 3D folded items; the unfolded virtual 3D item could be laid out on the same virtual 3D table on which the virtual 3D stack of folded 3D items appears, or could be worn by a virtual 3D mannequin displayed in close proximity to the virtual 3D table on which the virtual 3D stack of folded virtual 3D items appears.

The exemplary store owner may display certain items with a priority position on a virtual 3D display structure. For example, a store owner could position the newest virtual 3D model of a hairdryer at a front edge of a virtual 3D display shelf, and position the older models further back from the edge of the virtual 3D display shelf.

In an exemplary virtual store, some exemplary virtual items could be presented with enhancements to attract attention, such as in a case, where a particular manufacturer or provider has provided marketing incentives to the exemplary store owner. By way of illustrative, non-limiting example, a virtual 3D folded shirt by a particular manufacturer could be displayed as vibrating, or flashing, or expanding and then contracting, within a stack of shirts. As another example, a particular virtual 3D hardware item on a virtual 3D wall rack display could be specially lighted, or could vibrate, or flash. Such exemplary virtual effects would be provided as part of the above-mentioned exemplary store-owner virtual display design system.

In an exemplary virtual store, an exemplary shopping user could traverse the interior of the exemplary virtual store, such as by virtually moving through each virtual floor space, such as each exemplary isle, or each department area, within the exemplary virtual store. Alternatively, an exemplary shopping user could request a particular department, or search for a particular type of item, or search for a particular item. In one exemplary embodiment, such focused shopping could be done with voice input, or with text input, or with photo (of a type of item) input. The exemplary embodiment would use an artificial-intelligence-driven search engine to determine the best place in the virtual 3D store (or virtual 3D mall) for the exemplary shopping user to begin shopping.

When the exemplary shopping user had completed their shopping, the exemplary shopping user could then request check-out, such as, by way of illustrative, non-limiting example, clicking an exemplary virtual checkout option “button” on the user's exemplary virtual cart. (See e.g., exemplary virtual cart pay button element 1131 depicted in FIG. 12.) Alternatively, the exemplary shopping user could verbally announce that they are ready to check out.

Exemplary embodiments would provide store owners an opportunity to establish their own virtual store and would provide the store owners with the ability to specially curate, according to the store owner's marketing experience and reputation, the goods and brands to be presented for sale in their virtual 3D store. In some exemplary embodiments, although store owners could set up their own virtual 3D store as they wish, they would do so under the security (i.e., cybersecurity) provided by the mall owner or the administrator, as the case may be.

As compared to a two-dimensional list of pictures and item descriptions that seem to match an online shopping user's search, exemplary embodiments described herein would allow the shopping user to “walk” through a virtual 3D mall or store, browse various virtual 3D items of merchandise in various virtual departments, that have been curated in virtual 3D displays of items of similar types for various brands, and if a user is shopping for a particular type of item, to see the type of item they are shopping for in comparison to other items of a similar type. In such a digital analog to traditional three-dimensional physical shopping, the online shopping user would have the opportunity to see new styles and brands that the shopping user may find that they prefer over their historical preference. Further, by providing the store owners with the ability to curate displays and “enhance” or otherwise highlight, certain items, a shopping user may find items they had not previously realized they needed.

As will be understood by someone with ordinary skill in the art, other exemplary embodiments could be provided that would respond to a Shopping User's search for an item-such exemplary embodiments could fill a virtual room with a presentation of virtual items that correspond to the Shopping User's search, or could provide a “Guided Tour of Stores” that would present virtual displays of virtual items that correspond to the Shopping User's search, or that in other ways present virtual items that correspond to the Shopping User's search, without departing from the spirit of the present invention.

In an exemplary embodiment, each virtual shopping user's virtual presence and virtual location in a virtual 3D store (and/or a virtual 3D mall) would be shared with every other virtual shopping users that are virtually present (shopping online) in the virtual 3D store (and/or 3D mall) at the same time. Each virtual shopping user would be shown in the virtual 3D store/mall with respect to the virtual shopping user's location within the virtual 3D store; each virtual shopping user would be represented by a shopping avatar, or custom emoji, designed by the virtual shopping user, or as a Shopping Player GameObject that would be generated by some exemplary embodiments. In one exemplary embodiment, a particular virtual shopping user may choose to block or enable sharing of a view of their own virtual presence with other virtual shopping users virtually present in the virtual 3D store (and/or virtual 3D mall) at the same time. In one exemplary embodiment, a particular virtual shopping user may choose to block or enable viewing other virtual shopping users that are virtually present in the virtual 3D store and/or virtual 3D mall at the same time.

If the virtual shopping user chooses to view the other virtual shopping users present in the virtual 3D store (and/or virtual 3D mall) at the same time, it could give the virtual shopping user a feeling of community with people looking for similar items. In some exemplary embodiments, a particular virtual shopping user could “tap” (by way of non-limiting, illustrative example, clicking the emoji of another virtual shopping user) another virtual shopping user on the shoulder and ask them a question (by way of non-limiting, illustrative example, by text or voice, or by transcription of voice).

For example, as illustratively depicted in FIG. 4, a first shopping user (represented by first shopping user emoji 200) would be able to see a second shopping user (represented by second shopping user emoji 210) in a virtual location 220 in the virtual store. The first shopping user 200 would be able to “tap” the second shopping user 210 by, for example, placing the first shopping user's cursor 230 on the second shopping user's emoji 210 and clicking the cursor 230; the exemplary embodiment would then let the second shopping user know that the first shopping user had requested communication.

The illustrative exemplary embodiment would detect if two virtual shopping users attempt to communicate with each other in two different languages. For example, the illustrative exemplary embodiment could detect that two different languages were being used from each respective virtual shopping user's account settings for Primary Language. In the exemplary embodiment, a foreign language translator would automatically translate from one virtual shopper's language to another virtual shopper's language, in the event that the two virtual shoppers were to text or otherwise communicate with each other in two different languages. With reference to FIG. 16, such an exemplary tap 230 as depicted in FIG. 4, would activate the exemplary Online Shopping Marketplace 1520 to access 1590 the exemplary eCommerce Engine 1530 to access 1581 the exemplary Shopper Database 1580 to find language settings for the two shopping users 200, 210 depicted in FIG. 4 and compare their respective primary language settings; if the two exemplary shopping users had different primary languages set as their primary language, the secondary language settings would be checked; if no match is found between the primary and/or the secondary language settings for the two exemplary shopping users, then the exemplary embodiment would be programmed to cross-translate (text and/or voice).

As will be understood by someone with ordinary skill in the art, the herein described exemplary, automatic foreign language translation of cross-communications between two virtual users is illustrative and non-limiting; such exemplary, automatic foreign language translation of cross-communications between two virtual users in a common virtual space could be used in various settings outside of virtual shopping without departing from the spirit of the present invention. For example, cross-communications between two virtual users in other virtual settings would be possible, such as, for example, in virtual museums, in virtual art galleries, in virtual theaters, and the like.

In some illustrative exemplary mall embodiments of the present invention, shopping users (such as users that register with the administrator) would be provided with an exemplary fragrance emitting device embodiment of the present invention illustratively depicted in FIG. 5. As Illustratively depicted in FIG. 5, illustrative exemplary fragrance emitting device 400 would be communicatably connected 420 (such as, for example, by a USB port, Bluetooth, WiFi, or other wired or wireless communications whether now known or in the future discovered) with a computer device, such as, for example, a cell phone 410, and/or other computer devices whether now known or in the future discovered. The exemplary fragrance emitting device would in some ways be analogous to a color cartridge used with some printers. Instead of color being disbursed to a print surface, the exemplary fragrance emitting device 400 would emit certain fragrances as instructed by the computer device 410 to which the exemplary fragrance emitting device 400 would be communicatably connected 420. One illustrative, exemplary fragrance emitting device 400 would provide a plurality of fragrance pads 401a-401t for a plurality of fragrances of, such as, by way of illustrative, non-limiting example: rain, ice cream, brewing coffee, chocolate, popcorn popping, cut flowers, garlic sauteing, bacon frying, French fries frying, freshly mown grass, pizza baking, pharmacy clean, cinnamon rolls, yeast bread baking, sourdough baking, clean laundry, and other fragrances, including fragrances developed by individual store owners that signify an aspect of that individual store owner's virtual store.

The exemplary computer device 410 would receive an instruction from the exemplary Online Shopping Marketplace 1520 for emitting a particular fragrance and would in turn send an instruction to the exemplary fragrance emitting device 400 to activate a particular fragrance pad, e.g., exemplary fragrance pad 401a, at a particular level of fragrance intensity (e.g., mild, medium, or strong, or some particular fragrance intensity). Exemplary fragrance emitting device 400 would be programmed and configured to receive the exemplary instruction from the exemplary computer device 410 and to then send an exemplary fragrance pad activation signal to the particular fragrance pad 401a to emit a fragrance from fragrance pad 401a at an intensity according to the fragrance intensity instructed by the exemplary instruction from the exemplary computer device 410. In some exemplary embodiments, the exemplary instruction from the exemplary computer device 410 would further include a duration for fragrance emittance; the exemplary fragrance emitting device 400 would be programmed and configured to receive the instructed duration and send a fragrance emitting signal to fragrance pad 401a for the specified duration.

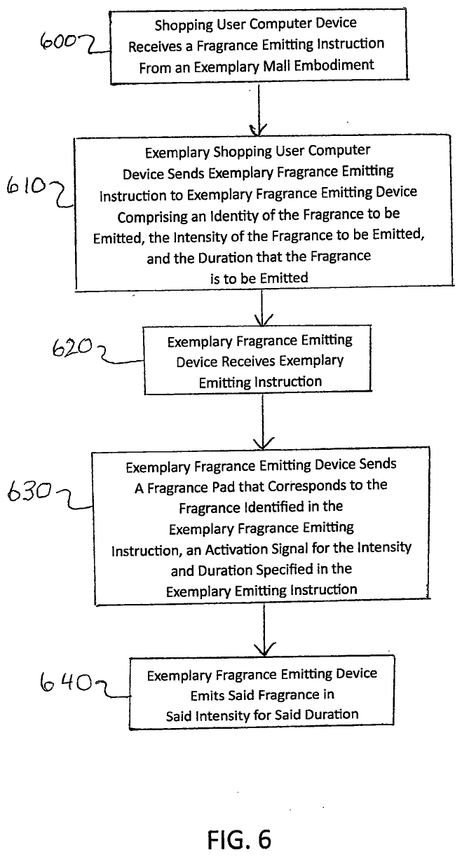

As further illustratively depicted in FIG. 6, the shopping user computer device would receive 600 a fragrance emitting instruction from an exemplary mall embodiment of the present invention.

The exemplary shopping user computer device would then send 610 an exemplary fragrance emitting instruction to an exemplary fragrance emitting device comprising an identity of the fragrance to be emitted, the intensity of the fragrance to be emitted, and the duration that the fragrance is to be emitted. The exemplary fragrance emitting device would receive 620 the exemplary emitting instruction. According to the exemplary emitting instruction, the exemplary fragrance emitting device would send 630 a fragrance pad that corresponds to the fragrance identified in the exemplary fragrance emitting instruction, an activation signal for the intensity and duration specified in the exemplary emitting instruction. The exemplary fragrance emitting device would then emit 640 said fragrance in said intensity for said duration.

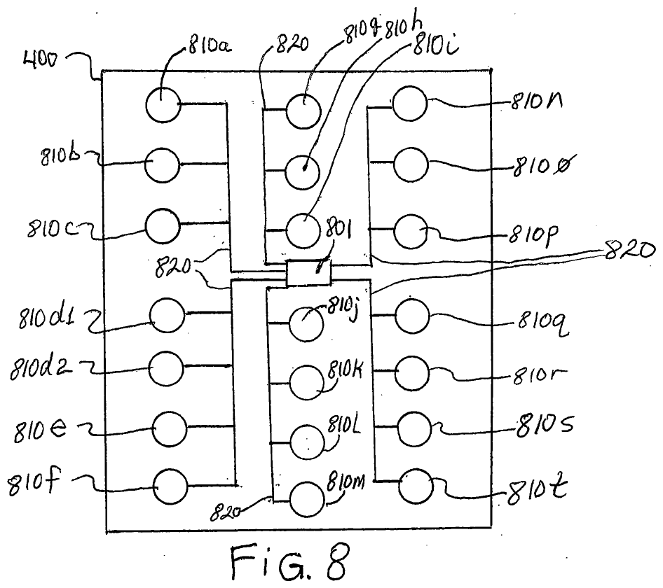

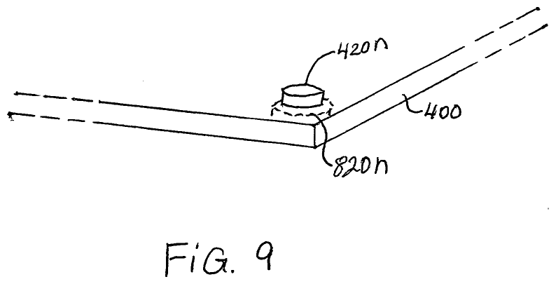

FIG. 8 illustratively depicts an exemplary diagram of components for an exemplary fragrance emitting device 400 in an exemplary embodiment of the present invention. FIG. 9 illustratively depicts an exemplary closeup view of an exemplary fragrance emitting element, e.g., 810n, that would set forth an exemplary fragrance pad 420n, in an exemplary fragrance emitting device 400 in an exemplary embodiment of the present invention.

As will be understood by someone with ordinary skill in the art, the exemplary depiction herein of the various components for an exemplary fragrance emitting device are illustrative and non-limiting. Other methods of implementing fragrance emitting elements and fragrance emitting computer devices would be possible without departing from the spirit of the present invention. For example, in other exemplary embodiments, fragrance emitting elements could be in various shapes, sizes, and numbers; they could sit above, or be imbedded in, or otherwise be set forth by, a further exemplary fragrance emitting device.

The exemplary fragrance emitting device 400 depicted in FIG. 8 would comprise an exemplary fragrance emitting computer device 801 that would comprise an exemplary wireless communication component for communicating with a shopping user's computer device. The exemplary fragrance emitting device 400 depicted in FIG. 8 would further comprise an exemplary plurality of fragrance emitting elements, e.g., 810a-810t, that would be connected to said exemplary fragrance emitting computer device 801. The exemplary fragrance emitting device depicted in FIG. 8 would further comprise a plurality of exemplary fragrances (e.g., exemplary fragrance elements (i.e., fragrance pads) 401a-401t as illustratively depicted in FIG. 5), wherein each exemplary fragrance of said plurality of exemplary fragrances would be contained in, or otherwise be set forth by, an exemplary fragrance emitting element of said plurality of fragrance emitting elements (e.g., 810a-810t).

The exemplary fragrance emitting computer device 801 depicted in FIG. 8 would be programmed to receive an instruction from a shopping user's computer device for emitting a particular fragrance. The exemplary fragrance emitting computer device 801 depicted in FIG. 8 would be further programmed to determine at least one fragrance emitting element of said plurality of fragrance emitting elements (e.g., 810a-810t) for emitting said particular fragrance. The exemplary fragrance emitting computer device 801 depicted in FIG. 8 would be further programmed to activate said at least one fragrance emitting element (e.g., one of 810a-810t) to emit a fragrance. The exemplary fragrance emitting computer device 801 depicted in FIG. 8 would be further programmed to activate at least one exemplary fragrance emitting element (e.g., one of 810a-810t) according to a particular intensity for a particular duration as would be instructed by the shopping user's computer device.

As will be understood by someone with ordinary skill in the art, there would be various ways to activate a fragrance emitting device, and there would be various forms in which the fragrances could be provided. By way of illustrative example, a manner of mildly heating a fragrance emitting element would cause a fragrance, such as in a wax form, or an oil form, to emit a fragrance. In some exemplary embodiments, instead of there being a one-to-one correspondence between a particular fragrance requested, and a particular fragrance emitting element, fragrances from a combination of fragrance elements (i.e., where each particular fragrance element would comprise elemental chemicals and/or elemental fragrance essences that could be combined to produce a particular fragrance) would comprise a particular fragrance.

In an exemplary embodiment where a plurality of fragrances would be combined to create a particular requested fragrance, the exemplary fragrance emitting computer device 801 depicted in FIG. 8, would be further programmed to determine each fragrance emitting element of said plurality of fragrance emitting elements (e.g., a subset of 810a-810t) needed for emitting said particular fragrance, and would simultaneously activate each determined fragrance emitting element according to a particular intensity for a particular duration.

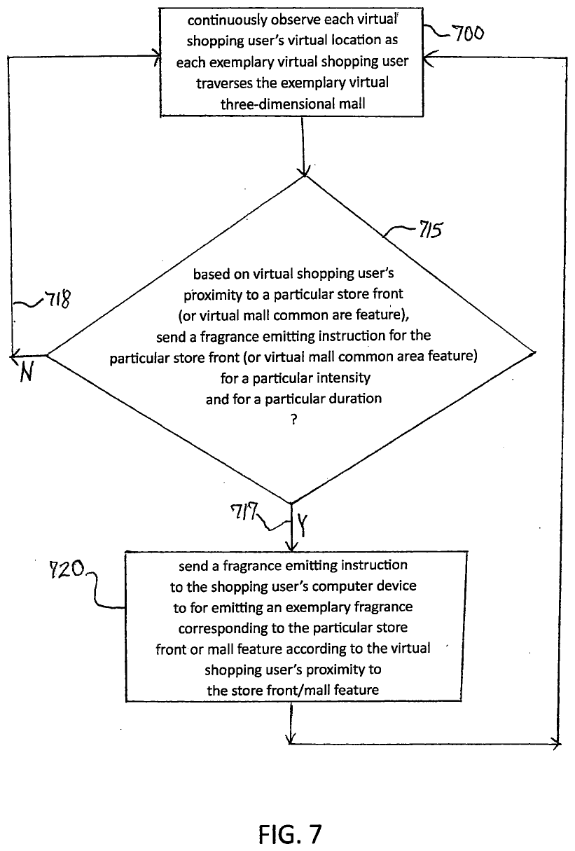

FIG. 7 illustratively depicts an exemplary high-level logic flow diagram of an exemplary mall embodiment of the present invention determining requirements for sending fragrance instructions for a particular fragrance to a particular virtual shopping user computer device. As an illustrative example use of such an illustrative, fragrance emitting device, as illustratively depicted in FIG. 7, the illustrative exemplary mall embodiment of the present invention would continuously observe 700 each virtual shopping user's virtual location as the exemplary shopping user traverses the illustrative, exemplary virtual three-dimensional mall. As the exemplary shopping user virtually traverses the illustrative, exemplary virtual three-dimensional mall embodiment of the present invention, the exemplary shopping user would virtually approach and then either pass by, or enter, various virtual storefronts and common area features. As the exemplary virtual three-dimensional mall embodiment continuously tracks each virtual shopping user's virtual location, it will continuously test the virtual shopping user's virtual location to determine 715 if the virtual shopping user is in proximity to a store front or common area mall feature for which it would be appropriate to send a fragrance emitting instruction, and based on the proximity, the intensity and duration for the fragrance emitting instruction. With reference to FIG. 16, the exemplary embodiment would do this by, for example, the exemplary Online Shopping Marketplace 1520 accessing 1590 the exemplary eCommerce Engine 1530 that in turn would access 1541 an exemplary Mall Database 1540 to obtain fragrance emitting information according to the shopping user's virtual location within the exemplary virtual mall. If the illustrative exemplary mall embodiment of the present invention determines 715, based on the store front (or mall feature) being approached, and the proximity of the virtual shopping user, that it should 717 send a fragrance emitting instruction for the particular store front, then the exemplary embodiment would send 720 a fragrance emitting instruction to the shopping user's computer device (the one that the shopping user is using to access the exemplary virtual mall) to emit an exemplary fragrance corresponding to the particular store front or mall feature according to the virtual shopping user's proximity to the store front/mall feature.

So, for example, if the exemplary virtual mall embodiment were to continuously track (700) the exemplary virtual shopping user's virtual location, and determine (e.g., 715 based on information from exemplary Mall Database 1540) that the virtual shopping user was approaching a virtual store front for a coffee shop for which the exemplary embodiment would send a fragrance emitting instruction, then the exemplary virtual mall embodiment would determine that it should send (e.g., 717), and would send (e.g., 720), a fragrance emitting instruction to the virtual shopping user's computer to emit a low intensity of an exemplary coffee brewing fragrance for some duration. The exemplary mall embodiment would continue to observe (e.g., 700) the virtual shopping user. As the shopping user begins to pass the virtual coffee shop, the illustrative exemplary mall embodiment would determine (e.g., 715) that it should (e.g., 717) send a fragrance emitting instruction to the shopping user's computer device to emit a stronger intensity of the exemplary coffee brewing fragrance and would send (e.g., 720) a fragrance emitting instruction to instruct the user's computer device to emit (for a particular duration) that stronger intensity.

As depicted in FIG. 7, if the exemplary virtual mall embodiment determines 715 that it should not send 718 any fragrance emitting instruction, then the exemplary virtual mall embodiment would continue to observe 700 the virtual shopping user's virtual location.

That is, as will be understood by someone with ordinary skill in the art, the illustrative exemplary mall embodiment would continuously track a shopping user's virtual movement through an exemplary virtual shopping space, such as through an exemplary virtual mall or virtual store, and would determine, based on the shopping user's virtual location within the exemplary virtual shopping space, whether or not to issue fragrance emitting instructions to the shopping user's computer device to instruct the exemplary fragrance emitting computer device associated with the shopping user's computer device to emit a particular fragrance, such as for a particular duration and at a particular intensity.

As will be understood by someone with ordinary skill in the art, the illustrative fragrances described herein are exemplary and non-limiting, other fragrances of various types would be possible in the spirit of the present invention and would add to a quality of realism for the virtual shopping user.

As will be further understood by someone with ordinary skill in the art, the herein described illustrative exemplary use of an exemplary fragrance emitting device is illustrative and non-limiting; other embodiments and alternative uses of exemplary fragrance emitting devices would be possible in the spirit of the present invention.

As will be further understood by someone with ordinary skill in the art, the herein described illustrative, exemplary fragrance emitting device 400 comprising a plurality of fragrance pads, e.g., fragrance pads 401a-401t for a plurality of fragrances is illustrative and non-limiting; instead of a plurality of fragrance pads, a palette of pads of fragrance elements could be provided, and an instruction to emit a particular fragrance would comprise a formula of fragrance elements to be combined to emit the particular fragrance.

As a further alternative, a palette of pads of chemicals could be provided, and an instruction to emit a particular fragrance would comprise a formula of chemicals to be emitted in a combined mixture to create the particular fragrance. In such a chemical palette embodiment of a fragrance emitting device, a first computer device would be programmed to communicate with a palette of a plurality of chemical reservoirs; the first computer device would be further programmed to receive a formula of chemicals, said formula of chemicals comprising a plurality of chemicals in said palette of a plurality of chemicals; wherein said formula of chemicals would further comprise a quantity of each chemical in said formula of chemicals; said first computer device further programmed to trigger said quantity of each chemical in said palette of a plurality of chemicals as represented in said formula of chemicals, said trigger comprising an instruction to jettison said quantity of said chemical into a mixture; and said first computer device further programmed to cause an emittance of said mixture, once said mixture is complete according to said formula, through a fragrance emitting device emitter. Alternatively, in some embodiments, the chemicals would be simultaneously emitted to combine to create the particular fragrance.

As will be further understood by someone with ordinary skill in the art, some exemplary embodiments of an exemplary fragrance emitter would comprise a combination of fragrance pads, fragrance reservoirs, fragrance element reservoirs, and/or chemical reservoirs without departing from the spirit of the present invention.

An exemplary mall embodiment of the present invention would be programmed to continuously monitor the location of each particular virtual shopping user, and to determine changes in the location of each particular virtual shopping user with respect to the timing of fragrance emitting; if a particular virtual shopping user virtually entered a particular virtual store in the virtual mall that corresponded to a particular fragrance just emitted for the particular shopping user, a small fee would be calculated that would be charged to the particular virtual store owner for the particular virtual store.

Some exemplary mall and store embodiments would continuously track and observe each virtual shopping user location as the shopping user traverses the virtual mall, or the virtual store, as the case may be, and would stream sounds to the shopping user's computer device that correspond to a feature, or features, that the virtual shopping user is approaching. For example, as the virtual shopping user approaches a mall common area fountain, the exemplary mall embodiment would stream the sound of cascading water to the shopping user's computer device. As the virtual shopping user approaches a mall common area playground, the exemplary mall embodiment would stream the sound of children playing to the shopping user's computer device. As the virtual shopping user approaches a plurality of other virtual shopping users, the exemplary mall embodiment would stream the sound of unperceivable conversation to the shopping user's computer device. As the virtual shopping user more closely approaches the feature for which there is a corresponding sound, the sound streamed would grow louder/stronger. As the virtual shopping user passes beyond a feature, the exemplary mall embodiment would stream a diminished sound of the feature. As will be understood by someone with ordinary skill in the art, the illustrative sounds described herein are exemplary and non-limiting, other sounds of various types would be possible in the spirit of the present invention and would add to a quality of realism for the virtual shopping user.

Some exemplary embodiments would continuously track and observe each virtual shopping user as the shopping user traverses the virtual mall, or the virtual store, or the virtual marketplace, as the case may be. Such observations could be recorded by such exemplary embodiments for a particular interactive virtual shopping session, or could be maintained in some exemplary embodiments for each virtual shopping user for use on return virtual shopping sessions by that particular virtual shopping user. The exemplary embodiment would record a tracking history of each virtual shopping user's locations and actions by the shopping user at each such location (such information would be stored, for example, in exemplary Shopper Database 1580 as illustratively depicted in FIG. 16). For example, if a virtual shopping user made a purchase within a virtual store, the exemplary embodiment would record that action in a corresponding tracking history for the virtual shopping user.

In some exemplary embodiments, as will be understood by someone with ordinary skill in the art, a purchase by a virtual shopping user at a virtual store during a virtual shopping session would be similar in some ways to a commonplace Internet online purchase—that is, the virtual shopping user would pay for items that were to be purchased from a particular virtual store and designate shipping information for the items. In an alternative exemplary virtual mall embodiment, a virtual shopping user would instead designate items in the virtual shopping user's cart be kept in an exemplary virtual mall locker; before leaving the virtual mall, the exemplary virtual shopping user would need to provide payment and shipping instructions for the items that the virtual shopping user wanted to purchase that were in the virtual shopping user's exemplary virtual mall locker. In some exemplary virtual mall embodiments, a virtual shopping user designating an item to be kept in an exemplary virtual mall locker would result in removal of the item from the stock of the particular virtual store. In other exemplary virtual mall embodiments, an item would not be removed from the stock of the particular virtual store unless and until the virtual shopping user provided payment and shipping information for the item.

If a virtual shopping user examined an item of merchandise in a virtual store, the exemplary embodiment would record that action in the corresponding tracking history for the virtual shopping user. If a virtual shopping user tarried in front of an item of merchandise in a virtual store, or a particular section of items of merchandise in a virtual store, the exemplary embodiment would record that action in the corresponding tracking history for the virtual shopping user. If a virtual shopping user tarried in front of a particular virtual storefront in the virtual mall, the exemplary embodiment would record that action in the corresponding tracking history for the virtual shopping user.

In the exemplary embodiment, certain actions by a virtual shopping user, and/or certain changes in location by a virtual shopping user, would initiate an analysis by the exemplary embodiment of the corresponding tracking history for the particular virtual shopping user. (E.g., the exemplary Online Shopping Marketplace 1520 illustratively depicted in FIG. 16 would access 1590 the exemplary eCommerce Engine 1530, illustratively depicted in FIG. 16, which would in turn access 1581 the relevant Shopper's tracking history from exemplary Shopper Database 1580 (as well as access 1541 exemplary Mall Database 1540, access 1561 exemplary Store Database 1560, and access 1551 exemplary Merchandise Item Database 1550) and provide 1590 that information back to the exemplary Online Shopping Marketplace 1520.) For example, if a particular virtual shopping user tarried in front of a particular section of items of merchandise in a first virtual store, but did not purchase any item from that particular section of items of merchandise, when the exemplary embodiment detects that the virtual shopping user has exited the first virtual store, the exemplary embodiment would analyze merchandise for other store owners in the virtual mall (or marketplace) to identify whether or not there are items of merchandise in other virtual stores in the same virtual mall that are similar to the items of merchandise that were in the particular section of that first virtual store.

If the exemplary embodiment identified one or more items of merchandise in other virtual stores in the same virtual mall that are similar to the items of merchandise that were in the particular section of that first virtual store, the exemplary embodiment would dynamically create an exemplary advertisement of the items for sale in one or more virtual stores. In some exemplary embodiments, the exemplary advertisement would be filtered to present an exemplary advertisement for the most similar item in a virtual store that carried that most similar item and had it in current stock; in other exemplary embodiments, various other types of filtering could be used, such as, for example, to identify the most similar items in a virtual store that also carries other items of possible interest for the particular virtual shopping user, based on an analysis by the exemplary embodiment of the particular virtual shopping user's shopping history.

Some exemplary advertisements created by an exemplary virtual mall embodiment would present an exemplary, visually rotatable, interactive, virtual three-dimensional representation of the item identified as similar to the virtual shopping user's interests, with exemplary detailed information about the item, such as a price and a particular virtual store identification and/or location (a “three-dimensional ad” embodiment); an exemplary virtual store location selection option would be presented in conjunction with the exemplary advertisement, that if selected by the virtual shopping user, would virtually transport the virtual shopping user through the exemplary virtual mall (which in some exemplary embodiments would be presented as if flying/zooming through the virtual mall; in other embodiments, it would be represented as an “elevate” and then descend at the appropriate store location; while in yet other exemplary embodiments it would be represented as a direct transport to the virtual store location) to the virtual store location of a virtual storefront for the virtual store.

In an exemplary three-dimensional ad embodiment, an exemplary, visually rotatable, interactive, virtual three-dimensional representation of the item to be advertised would be dynamically generated and presented in virtual mid-air, in virtual, visual proximity to the location of the virtual shopping user.

Other exemplary embodiments would generate two-dimensional advertising, such as, for example, a virtual poster with a picture of the item and additional detail about, for example, the price and the particular virtual store identification and/or location (a virtual “two-dimensional ad” embodiment). In such an exemplary, virtual two-dimensional ad embodiment, a generated virtual two-dimensional ad would be presented to the subject virtual shopping user, by, for example, mounting the two-dimensional ad, on a wall, or on an advertising sign, in the interior of the exemplary virtual mall.

In both the two-dimensional and three-dimensional exemplary ad embodiments, the exemplary virtual store location selection option would be presented in conjunction with the exemplary advertisement, that if selected by the virtual shopping user, would cause the exemplary embodiment to virtually transport the virtual shopping user through the exemplary virtual mall to the virtual store location of the virtual storefront for the virtual store that offers the advertised item for sale.

In a manner analogous to the elsewhere herein emitting of a fragrance, as a virtual shopping user approaches a particular virtual storefront, the exemplary embodiment would identify items of possible interest based on the shopping history of the virtual shopping user that would be available in the virtual store associated with the particular virtual storefront. The exemplary embodiment would dynamically create and begin presenting to the virtual shopping user as that user begins virtually approaching the virtual storefront, in either a two-dimensional, or a three-dimensional, form, an advertisement about one or more items of possible interest that would be available for sale in the approaching virtual store.

In an exemplary beginning presentation of the above-mentioned approaching-storefront advertisement, the exemplary advertisement would present directional indicators, e.g., arrows, pointing to the approaching virtual storefront.

As the virtual shopping user more closely virtually approaches the particular virtual storefront, the exemplary embodiment would intensify the appearance of the advertisement, such as, for example, with more intense coloration, more intense lighting, a larger size, or the like.

After an exemplary advertisement is presented to a particular virtual shopping user that is approaching a particular virtual storefront, if the particular virtual shopping user enters the particular virtual store that is associated with the particular virtual storefront, then the exemplary embodiment would charge the owner of the particular virtual store with an exemplary successful approach advertising fee.

After an exemplary advertisement with an exemplary virtual store location selection option is presented to a particular virtual shopping user, if that particular, virtual shopping user subsequently selects the exemplary virtual store location selection option and subsequently enters the virtual store that had been identified in the exemplary advertisement, then the exemplary embodiment would charge the owner of the particular virtual store with an exemplary successful store entry advertising fee.

After an exemplary advertisement with an exemplary virtual store location selection option is presented to a particular virtual shopping user, if that particular, virtual shopping user subsequently purchases the item featured in the exemplary virtual advertisement, from the location identified, and the virtual store named, in the exemplary advertisement, then the exemplary embodiment would charge the owner of the virtual store named in the exemplary embodiment with an exemplary successful advertising fee.

If, on the other hand, that particular, virtual shopping user subsequently inspects, but does not purchase, the item featured in the exemplary virtual advertisement, but instead purchases another item at the location identified, and in the virtual store named or otherwise identified, in the exemplary advertisement, then the exemplary embodiment would charge the owner of the virtual store named or otherwise identified in the exemplary embodiment with an exemplary effective advertising fee.

Inside a particular virtual store, similar approaches to presenting virtual, exemplary in-store advertisements would be provided by some exemplary ad embodiments. For example, as a virtual shopping user traverses a particular virtual store, as the virtual shopping user approaches a particular virtual department within the particular virtual store, the exemplary embodiment would present exemplary approach advertisements for the approaching exemplary virtual department, such as for particular items that would be of possible interest to the virtual shopping user based on the virtual shopping user's shopping history within the particular virtual store.

Further, once a particular virtual shopping user enters a particular virtual store, based on that virtual shopping user's shopping history within that particular virtual store, some exemplary embodiments would present sales going on within the exemplary virtual store of items of possible interest to the virtual shopping user, with an exemplary virtual item, or department, location selection option.

Some exemplary ad embodiments would be programmed with an exemplary artificial-intelligence-driven merchandise-to-user-shopping-history analyzer. The exemplary artificial-intelligence-driven merchandise-to-user-shopping-history analyzer would be trained to recognize items with “similarities” to items that a particular virtual shopping user had previously purchased or examined, and would be further trained to identify items that would potentially complement items that the virtual shopping user had either purchased or examined. As an illustrative, non-limiting example, if a particular shopping user had previously, recently purchased a long evening gown, the exemplary artificial-intelligence-driven merchandise-to-user-shopping-history analyzer would have been trained to identify long underslips as a potentially complementing item of potential interest to the virtual shopping user. As another illustrative, non-limiting example, if a particular virtual shopping user had previously purchased electrical tools, the exemplary artificial-intelligence-driven merchandise-to-user-shopping-history analyzer would have been trained to identify work gloves as a potentially complementing item of potential interest to the virtual shopping user.

In some exemplary embodiments, shopping users would be given the option to view, or not view, generated advertisements; electing to view the generated advertisements could earn the shopping user discounts.

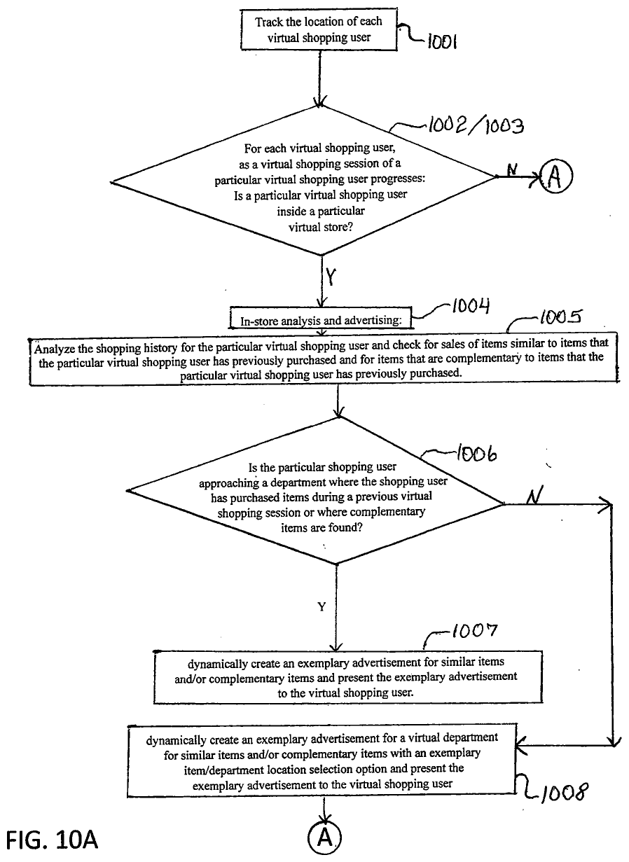

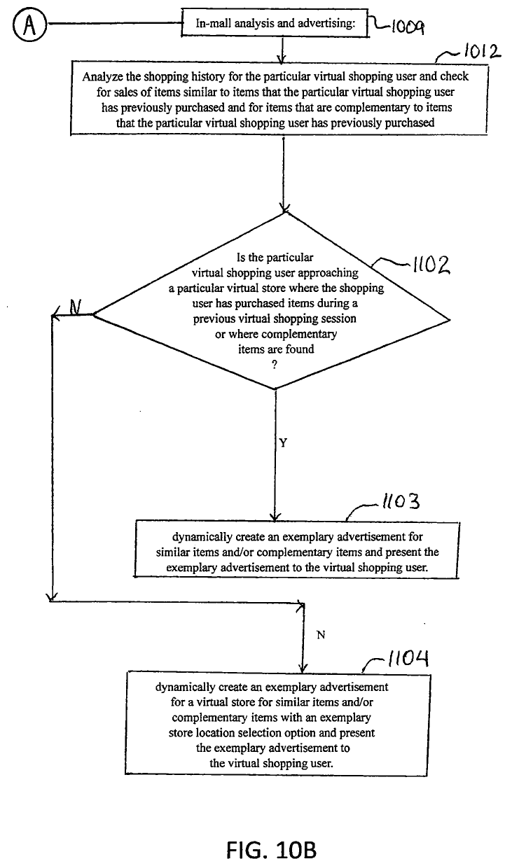

FIGS. 10A-10B present a high-level logic flow diagram depicting an exemplary advertising logic flow in an exemplary ad embodiment of the present invention. As depicted in FIGS. 10A-10B the exemplary ad embodiment would track 1001 the location of each virtual shopping user. As further depicted in FIGS. 10A-10B, for each virtual shopping user 1002, as a virtual shopping session of a particular virtual shopping user progresses, the exemplary ad embodiment would determine 1003 whether a particular virtual shopping user is inside a particular virtual store or out in the common areas of the mall. If a particular virtual shopping user is determined to be inside a virtual store, then the exemplary ad embodiment would then perform in-store analysis and advertising 1004. If, on the other hand, the exemplary ad embodiment determines that the particular virtual shopping user is in a marketplace common area (i.e., outside of virtual stores), then the exemplary ad embodiment would perform in-mall analysis and advertising 1009.

In the exemplary ad embodiment, in-store analysis and advertising 1004 would comprise, for example:
Analyzing 1005 the shopping history for the particular virtual shopping user and checking for sales of items similar to items that the particular virtual shopping user has previously purchased and/or inspected, and for items that are complementary to items that the particular virtual shopping user has previously purchased. In the exemplary embodiment, in order to perform exemplary shopping history analysis (both In-store and In-mall), as illustratively depicted in FIG. 16, the exemplary Online Shopping Marketplace 1520 illustratively depicted in FIG. 16, would access 1590 the exemplary eCommerce Engine 1530, illustratively depicted in FIG. 16, which would in turn access 1581 the relevant Shopper's tracking history from exemplary Shopper Database 1580 (as well as access 1541 exemplary Mall Database 1540, access 1561 exemplary Store Database 1560, and access 1551 exemplary Merchandise Item Database 1550); the exemplary eCommerce Engine 1530 would in turn provide 1590 the relevant information back to the exemplary Online Shopping Marketplace 1520;
Determining 1006 whether the particular virtual shopping user is approaching a department where the shopping user has purchased and/or inspected items during a previous virtual shopping session or where complementary items are found;
   If so, dynamically creating 1007 an exemplary advertisement for similar items and/or complementary items and presenting the exemplary advertisement to the virtual shopping user;
   If not, dynamically creating 1008 an exemplary advertisement for a virtual department for similar items and/or complementary items with an exemplary item/department location selection option and presenting the exemplary advertisement to the virtual shopping user.

In some exemplary ad embodiments, In-mall analysis and advertising 1009 would comprise, for example:
Analyzing 1012 the shopping history for the particular virtual shopping user and checking for sales of items similar to items that the particular virtual shopping user has previously purchased and/or inspected, and for items that are complementary to items that the particular virtual shopping user has previously purchased;
Determining 1102 whether the particular virtual shopping user is approaching a particular virtual store where the shopping user has purchased and/or inspected items during a previous virtual shopping session, or where complementary items are found;
   If so, dynamically creating 1103 an exemplary advertisement for similar items and/or complementary items and presenting the exemplary advertisement to the virtual shopping user;
   If not, dynamically creating 1104 an exemplary advertisement for a virtual store for similar items and/or complementary items with an exemplary store location selection option, and presenting the exemplary advertisement to the virtual shopping user.

### Exemplary Massively Multi-Player Online Shopping Game (Mmosg) Embodiments

Some exemplary embodiments of the present invention would comprise apparatus, systems and methods for creating, activating, displaying in a computer display (whether two-dimensional or virtual-reality), and interacting with, a shared, interactive shopping session in a persistent, virtual three-dimensional massively multi-player online game (MMOG) integrated with a back-end electronic commerce engine that would have access to a plurality of Databases (see, e.g., FIG. 16). As mentioned elsewhere herein, some exemplary embodiments would be provided as part of a Massively Online Shopping Game (“MMOSG”).

As will be understood by someone with ordinary skill in the art, the exemplary discussion herein of exemplary display in a two-dimensional computer display is not a limitation of the present invention. Indeed, virtual reality three-dimensional gaming technology exists that would be suitable for display and interaction with exemplary embodiments of the present invention. However, at the time of the writing of this patent application, such virtual reality three-dimensional gaming technology is not yet in the hands of many people who could nevertheless benefit from and/or enjoy shopping using exemplary embodiments of the present invention. As will be understood by someone with ordinary skill in the art, other exemplary embodiments of the present invention would be provided to display and interact with an exemplary Front-End of the present invention on such virtual three-dimensional gaming technology without departing from the spirit of the present invention.

As will be understood by someone with ordinary skill in the art, some game engines, such as, for example, Unity software, are object-oriented. As will be further understood by someone with ordinary skill in the art, in an exemplary Unity-software-based embodiment, GameObjects would be fundamental objects to which components can be assigned and programmed to provide functionality, interactivity characteristics, and movement, for the GameObject.

As will be understood by someone with ordinary skill in the art, game engines, such as, for example, Unity software, have been used to create games that users play with other players (sometimes with a massive number of other players)—the games may provide adventures, puzzles, or other online gaming features, within the gaming space (local or global).

As compared to playing a game merely within a gaming space, exemplary embodiments of MMOSG embodiments would provide a “Front-End” “game” that would allow Playing Users to shop for virtual representations of real-life items of merchandise that would be virtually displayed as virtual 3D Merchandise Item “GameObjects” in the exemplary game space; the virtual display of real items of merchandise as virtual 3D Merchandise Item “GameObjects,” would be linked to exemplary databases (e.g., such as the exemplary Merchandise Item 1550 illustratively depicted in FIG. 16) that would contain information about the real-life items of merchandise so that a Playing User would be able to purchase the real-life item of merchandise by using a Player Emoji (or Player GameObject) controlled by the Playing User to place a virtual 3D Merchandise Item “GameObject” in the Playing User's virtual cart and by then completing a purchase transaction for that virtual 3D Merchandise Item “GameObject” item.

As will be understood by someone with ordinary skill in the art, in some exemplary MMOSG embodiments, instead of an exemplary Playing User controlling a Playing Emoji, the exemplary Playing User would be able to shop as what is sometimes referred to in the gaming world as a “First Person Shooter,” (“FPS”), but which would be understood as being a “First Person Shopper” in exemplary MMOSG embodiments. In an FPS MMOSG embodiment, instead of a Playing User controlling the actions and movement of a Playing Emoji, a Playing User would see the “Hands” of the Playing User Object and would control the movement and actions of the “Hands.”

In an exemplary Massively Multiplayer Online Game embodiment (as compared to typical Online eCommerce Shopping two-dimensional lists presented in response to a user's search request), each Playing User would be able to “see” all of the other Playing Users (with their respective permission) that are currently shopping in the same virtual department of the same virtual store in which the exemplary Playing User would be shopping. Therefore, in exemplary MMOSG embodiments, whether an exemplary Playing User views themselves as their own hands (such as controlling their virtual cart, as in a First Person Shopper/Shooter FPS Game), or as their Playing Emoji, other exemplary Playing Users will “see” them (the Playing User) as their (the Playing User's) Playing Emoji.

Some exemplary MMOSG embodiments would further provide an exemplary “Back-End” that would comprise programming software and one or more exemplary databases. In some exemplary MMOSG embodiments, each virtual 3D merchandise [GameObject] item would be digitally linked to one or more exemplary Merchandise Item database entry/ies (e.g., such as in the exemplary Merchandise Item Database 1550 illustratively depicted in FIG. 16) that would provide, by way of non-limiting example, merchandise item specifications, such as, as the case may be: Brand information, such as a Brand name; item description; item price; measurements; color; size; 3D dimensions; proportionally scaled 3D dimensions (for virtual display on virtual supporting-structure GameObjects); virtual 3D location information; manufacturer information; operating specifications; installation information; assembly instructions; material composition information; UPC barcode; ISBN barcode; inventory information; supplier information; virtual store information; inventory information; shipping information; and other types of merchandise item information.

In some exemplary MMOSG embodiments, exemplary virtual supporting-structure GameObjects would be provided for virtually housing, supporting, displaying, and/or presenting exemplary virtual 3D merchandise items. By way of non-limiting example, exemplary shelves, cases, racks, mannequins, and/or other exemplary virtual supporting-structure GameObjects would be provided. In some exemplary embodiments, information regarding such exemplary virtual supporting-structure GameObjects would be stored in an exemplary Back-End Database (such as, e.g., exemplary Support Structure Database 1570 illustratively depicted in FIG. 16.) Exemplary virtual 3D Merchandise Item GameObjects would be arranged on exemplary virtual supporting-structure GameObjects for presentation to virtual online Shoppers.

In some exemplary MMOSG embodiments, some exemplary MMOSG Back-End Item database information would further comprise, for example, GameObject Component Information, including, but not limited to: GameObject component types associated with an exemplary particular virtual 3D merchandise item; GameObject component settings for each GameObject component type associated with the exemplary particular virtual 3D merchandise [GameObject] item; and component scripts for each GameObject component type associated with the exemplary particular virtual 3D merchandise [GameObject] item.

In some exemplary MMOSG embodiments, each virtual 3D Player GameObject would be digitally linked to one or more exemplary 3D Player database entries (e.g., such as the exemplary Shopper Database 1580 illustratively depicted in FIG. 16) that would provide, by way of non-limiting example, exemplary virtual 3D Player information, such as, as the case may be: Player Name; Player Account Information (such as, by way of non-limiting example: Account Number; User Identification information; User Password; User Biometric Information; Player Emoji Information); Player Gaming Name; Player address; Player contact information (e.g., telephone number, email address, etc.); Player payment information; Player shopping attributes; Player preferences (such as, by way of non-limiting example: Store preferences; department preferences; brand preferences; size preferences; color preferences (which may be differentiated by shopping item type, such as for clothing types; furniture; appliances; material preferences; appliance size preferences; appliance operational preferences; etc.

Exemplary Player Emoji Information may comprise, by way of non-limiting example, including among others: Emoji [GameObject] appearance; Emoji [GameObject] dimensions; Emoji [GameObject] components; Emoji [GameObject] movement characteristics (e.g., Idle scripting; reaching scripting; item grabbing scripting; item examining scripting; item placement in cart scripting; item removal from cart scripting; movement scripting; and the like), etc.

Some exemplary MMOSG embodiments would provide a virtual MMOSG mall embodiment, wherein a virtual 3D mall would comprise a plurality of virtual 3D stores and a plurality of virtual 3D common areas. In such an exemplary virtual MMOSG mall embodiment, the exemplary mall would be linked to an exemplary Mall database entry (e.g., such as an entry in an exemplary Mall database 1540 as illustratively depicted in FIG. 16) that would comprise, by way of illustrative, non-limiting examples, a plurality of data components that would describe the exemplary virtual mall, including among others: mall layout; mall dimensions; store layout within the virtual mall; store account information; common area feature layout; common area feature information; etc.

In some virtual MMOSG mall embodiments, each virtual 3D store would be linked to an exemplary Store database entry (e.g., such as an entry in an exemplary Store database 1560 as illustratively depicted in FIG. 16) that would comprise, by way of illustrative, non-limiting examples, a plurality of data components that would describe the exemplary virtual store, including among others: virtual 3D store layout; virtual 3D store dimensions; virtual 3D department layout within the virtual 3D store; virtual supporting-structure GameObjects layout within the virtual 3D store department; store account information; store owner information; store owner address; store owner contact information including phone numbers, fax numbers, email addresses and the like; etc.

In some virtual MMOSG embodiments, all sensitive information (such as personal identifying information, account information and payment information) would be kept in an exemplary secure digital vault (e.g., such as in an exemplary Shopper database 1580 as illustratively depicted in FIG. 16); access to information in such a secure digital vault would involve, for example, an exemplary encryption of a plurality of data components from the relevant Player's or Store's data in order to access the sensitive information in the exemplary secure digital vault.

Each Playing User in an exemplary MMOSG embodiment would be provided with a Player Emoji and would be able “shop” in the MMOSG Shopping Game through the Playing-User-Controlled movements of the Player Emoji. A Playing User would control the movement of the Playing User's Player Emoji through the exemplary MMOSG mall and through each MMOSG store, and within an MMOSG store, through exemplary departments within an exemplary MMOSG store.

An exemplary Playing User would control the exemplary movement and actions of the exemplary Playing User's Player Emoji using, for example, directional arrows or other directional keys on a keyboard, or using a mouse, or keyboard touchpad, or other directional controls, whether now known or in the future discovered, or some combination thereof. Each exemplary MMOSG embodiment would define Player Emoji directional controls and would teach each exemplary Playing User to use them to control the movement and actions of the exemplary Playing User's Player Emoji.

A number of illustrative figures are provided to depict non-limiting, exemplary shopping scenarios. For example, FIG. 11 illustratively depicts an exemplary Shopping User/Shopping Player Emoji 1010 as an exemplary Online Computer Shopper Emoji entering an exemplary virtual Mall through an exemplary virtual Mall entrance 1020 in an exemplary MMOSG mall embodiment. FIG. 12 illustratively depicts an exemplary Shopping User's/Shopping Player's Shopping Emoji 1010 in an exemplary Mall in front of an exemplary virtual Store Front 1110; exemplary shopping mall sounds 1120 would be heard by the exemplary online shopper through exemplary audio streaming to the online shopping user's computer device; the Shopping Emoji 1010 could grab an exemplary mall Shopping Cart 1130.

FIG. 13 illustratively depicts an exemplary Shopping User's/Shopping Player's Shopping Emoji/GameObject 1010 shopping in an exemplary virtual Store 1210; exemplary Store sounds 1220 would be heard by the exemplary online shopper through exemplary audio streaming to the online shopping user's computer device based on the virtual location of the exemplary Shopping User's/Shopping Player's Shopping Emoji/GameObject 1010 in the exemplary virtual Store 1210; other virtual Shopping Users'/Shopping Players' Shopping Emojis/GameObjects, e.g., 1250, 1270, that are shopping in the same virtual area of the virtual exemplary Store 1210 as the exemplary Shopping User's/Shopping Player's Shopping Emoji/GameObject 1010, would be visible to each other; each exemplary Shopping User/Shopping Player would be able to hear the sound effects of, and see, the exemplary placement of the exemplary virtual three-dimensional item of merchandise in the Shopping Emoji's virtual cart 1130 (See, e.g., FIG. 17, the exemplary Shopping Users Shopping Emoji 1010 in the placing exemplary Merchandise Item GameObject 1740 in the Shopping Emoji's virtual cart 1130). The exemplary Shopping User's/Shopping Player's Shopping Emoji/GameObject would be able to browse exemplary virtual items of merchandise, e.g., 1231-1236, and 1241-1243, illustratively virtually displayed on exemplary virtual display supporting structures 1230, 1240.

FIG. 14 illustratively depicts an exemplary Shopping User's/Shopping Player's Shopping Emoji/GameObject 1010 viewing a plurality of exemplary virtual three-dimensional items of merchandise 1241-1243 displayed on an exemplary virtual table 1240. FIG. 15 illustratively depicts the exemplary Shopping User's/Shopping Player's Shopping Emoji/GameObject 1010 touching 1510 (sometimes elsewhere herein referred to as a “touch selection”) one of the plurality of exemplary virtual three-dimensional items of merchandise 1242 displayed on the exemplary virtual table 1240; touching (e.g., 1510) the exemplary item and/or holding the touch (e.g., 1510) is illustratively depicted to cause an exemplary interactive display 1410 of information about the item, such as, by way of non-limiting, illustrative example, among other things: price, sizes available, colors available, manufacturer, material, where made, and the like. FIG. 16 illustratively, graphically depicts an exemplary shared Online Shopping Marketplace 1520 illustratively interacting 1590 with an exemplary eCommerce Engine 1530 in response to an Online Virtual Shopper Emoji 1010 illustratively touching 1510 exemplary virtual Merchandise Item 1242; the exemplary eCommerce Engine 1530 would then, in turn, access (1541, 1551, 1561, 1571 and 1581) a number of various databases, e.g., 1540-1580 of information relevant to the Online Shopping Marketplace 1530; in FIG. 15, an exemplary trigger 1510 for the exemplary shared Online Shopping Marketplace 1520 to illustratively interact 1590 with the exemplary eCommerce Engine 1530 is depicted in response to the exemplary Shopping User's/Shopping Player's Shopping Emoji/GameObject 1010 virtually touching (1510) an exemplary virtual item of merchandise 1242. FIG. 17 illustratively depicts the exemplary Shopping User's/Shopping Player's Shopping Emoji/GameObject 1010 leaving the exemplary virtual table 1240 displaying the plurality of exemplary virtual three-dimensional items of merchandise 1241-1243 with an empty virtual shopping cart 1130. Among other things, FIG. 16 illustratively depicts exemplary Merchandise Item Database 1550 being accessed 1551 by exemplary eCommerce Engine 1530 in response to exemplary access 1590 by exemplary Online Shopping Marketplace 1520 in response to exemplary shopping user 1010 illustratively touching 1510 exemplary virtual Item of Merchandise 1242.

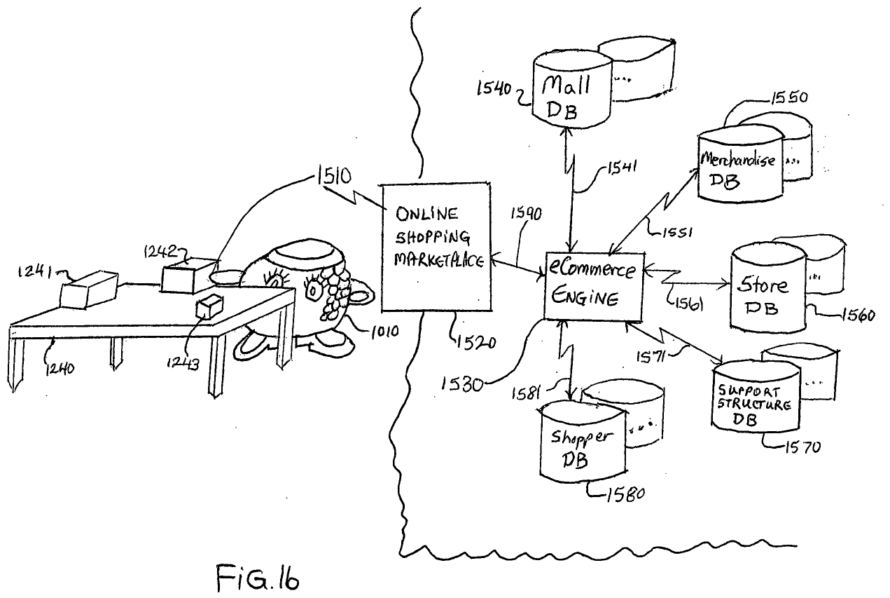

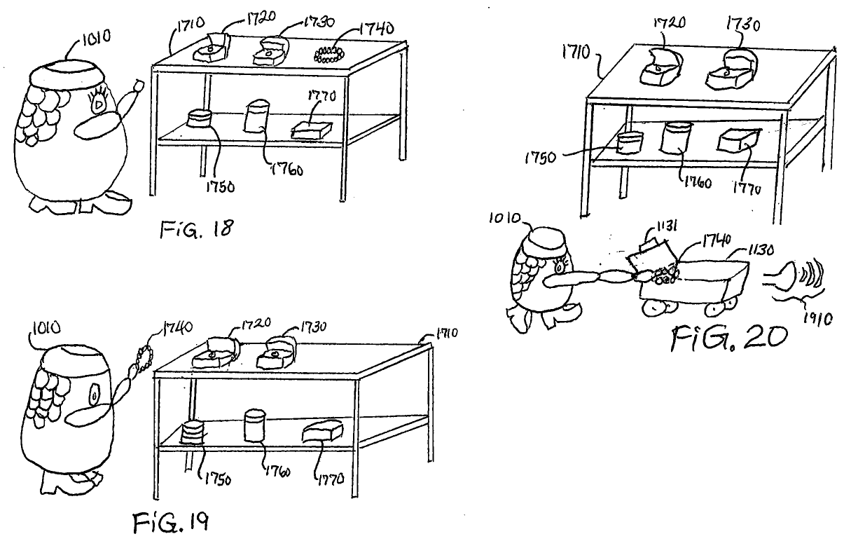

FIG. 18 illustratively depicts an exemplary Shopping User's/Shopping Player's Shopping Emoji/GameObject 1010 approaching an exemplary virtual table 1710 that virtually displays a plurality of exemplary virtual three-dimensional items of merchandise 1720-1770. Exemplary embodiments would detect an interactive virtual manipulation selection by a particular Online Shopping Player of an interactive, virtual, three-dimensional Merchandise Item GameObject. In some exemplary embodiments, such as those that are accessed with a two-dimensional computer display, keyboard keys, such as arrow keys, could be used to signal the exemplary embodiment to rotate a virtual item (e.g., rotate left, rotate right, rotate up, rotate down). In exemplary embodiments that would be accessed by an online shopper using a virtual-reality display and accessories, various hand commands would be interpreted by the exemplary embodiment to virtually rotate the display of the interactive, virtual, three-dimensional Merchandise Item GameObject. Whether accessed by virtue of a two-dimensional computer display and attending keyboard (or mouse) accessories, or accessed by a virtual-reality display and attending accessories, exemplary embodiments would respond to an interactive virtual manipulation selection by a particular online shopping player with a corresponding display of a virtual presentation of the interactive, virtual, three-dimensional Merchandise Item GameObject; and would respond to interactive virtual manipulation instructions by the particular player of the interactive, virtual, three-dimensional game object by displaying virtual rotation of the interactive, virtual, three-dimensional Merchandise Item GameObject according to the interactive virtual manipulation instructions provided by the particular player. FIG. illustratively depicts the exemplary Shopping User's/Shopping Player's Shopping Emoji/GameObject 1010 examining a particular exemplary virtual three-dimensional item of merchandise 1740 that the exemplary Shopping Emoji picked up from the exemplary virtual table 1710. As elsewhere mentioned herein, in exemplary embodiments, each virtual item of merchandise would be visually rotatable—that is an online shopping user would be able to virtually select an exemplary virtual item of merchandise (e.g. 1740) (for manipulation), and would be able to virtually turn it around (virtually manipulate it) to see it from various angles, before deciding whether or not to put the virtual item in the online shopper's cart, or put it back on its respective display.

Exemplary embodiments would respond to an interactive “keep” selection by a particular online shopping player of a particular virtual, interactive, three-dimensional Merchandise Item GameObject by displaying a virtual placement of the particular virtual, interactive, three-dimensional game object in an interactive, virtual, three-dimensional cart game object. FIG. 20 illustratively depicts the exemplary Shopping User's/Shopping Player's Shopping Emoji/GameObject 1010 placing the particular exemplary virtual three-dimensional item of merchandise 1740 in the Shopping Emoji's virtual cart 1130; exemplary sound effects 1910 are depicted as being heard from the exemplary placement of the exemplary virtual three-dimensional item of merchandise 1740 in the Shopping Emoji's virtual cart 1130.

Exemplary embodiments would respond to an interactive “pay” selection (by way of non-limiting example, a double-click of a cursor in a two-dimensional access scenario; or a double tap of the item in a virtual-reality access scenario) by a particular player of a particular virtual, interactive, three-dimensional Merchandise Item GameObject by engaging a digital link (if the exemplary embodiment had not already done so) associated with the particular virtual, interactive, three-dimensional game object in order to interactively access (if the exemplary embodiment had not already done so) a set of data stored in a database (e.g., such as in the exemplary Merchandise Item Database 1550 illustratively depicted in FIG. 16) where the set of data would comprise, among other things, a price. Exemplary embodiments would further respond to such an interactive “pay” selection by charging an account associated with the particular player for the amount of the price associated by the set of data with the interactive, virtual, three-dimensional Merchandise Item GameObject. Alternatively, in some exemplary embodiments, a virtual online shopping player could press an exemplary “pay” button, such as exemplary “pay” button 1131 on exemplary virtual cart 1130 as illustratively depicted in FIG. 20 to request payment for all items in the particular online shopping user's virtual cart 1130.

In some exemplary MMOSG embodiments, “gaming” features would be employed to elevate a shopping user's excitement for shopping. By way of illustrative, non-limiting examples of such “gaming” features, playful sound effects would be possible, such as when a shopping user places an item in the shopping user's cart (see, e.g., FIG. 19); another type of playful sound effects would be possible when a shopping user returns an item to its display location (see, e.g., FIG. 16); online shoppers in some embodiments would be able to “purchase” special virtual clothing and/or accessories for their shopping emojis or shopper GameObjects; and in some embodiments, online shoppers could pay extra to be represented by personalized, customized shopping emojis or shopper GameObjects.

As further illustrative, non-limiting “gaming” feature examples, some exemplary embodiments would allow a shopping user to elect to turn on certain gaming elements to earn credits or special discounts for purchases, such as, for example, by: maneuvering through, over, and/or around virtual physical impediments or barriers on approaching a special sale display of, and/or manipulating certain physical features in the virtual store landscape in order to access, virtual items of merchandise, and/or, outsmarting virtual dynamic energy features that attempt to prevent placement of a virtual item of merchandise in the shopping user's cart.

In some exemplary MMOSG embodiments, when the exemplary embodiment receives 3D scan data for various items (e.g., of merchandise, or displays), the exemplary embodiment would convert the exemplary 3D data to exemplary Low Poly [gon] virtual 3D images; some exemplary embodiments would use Artificial-Intelligent-programmed software to convert the 3D scan data to such exemplary Low Poly [gon] virtual 3D images and would further use such AI-programmed software to arrange such exemplary Low Poly [gon] virtual 3D images on exemplary virtual supporting-structure Display GameObjects for virtual display to online shopping users.

What is claimed is:

1. A computer system programmed to:
Generate an online interactive, virtual, three-dimensional session distributable to a plurality of player computer devices and displayable on a plurality of computer displays, wherein each computer display of said plurality of computer displays is associated with a particular player computer device of said plurality of player computer devices;
Generate an interactive, virtual, three-dimensional merchandise item game object in said online interactive, virtual, three-dimensional session, said interactive, virtual, three-dimensional merchandise item game object corresponding in virtual three-dimensional relationship characteristics to a physical item of merchandise for sale; and
Digitally link said interactive, virtual, three-dimensional merchandise item game object to a set of data specific for said interactive, virtual, three-dimensional merchandise item game object, said set of data stored in a database, said database accessible by said computer system, said set of data comprising specifications about said interactive, virtual, three-dimensional merchandise item game object, said set of data comprising a price to purchase said physical item of merchandise corresponding to said interactive, virtual, three-dimensional merchandise item game object.

2. The computer system of claim 1, said computer system further programmed to:
Detect an interactive touch selection by a particular player of said online interactive, virtual, three-dimensional session of said interactive, virtual, three-dimensional merchandise item game object, wherein said interactive touch selection comprises a request for information about said interactive, virtual, three-dimensional merchandise item game object received by said computer system from a particular player computer device used by said particular player to interact with said online interactive, virtual, three-dimensional session;
Respond to said interactive touch selection by said particular player of said interactive, virtual, three-dimensional merchandise item game object by interactively accessing said set of data stored in said database, said set of data comprising a price associated with said interactive, virtual, three-dimensional merchandise item game object; and
Display said price to a computer display associated with the particular player computer device used by said particular player to interact with said online interactive, virtual, three-dimensional session, said display said price displayed interactively in association with said interactive, virtual, three-dimensional merchandise item game object.

3. The computer system of claim 1, said computer system further programmed to:
Detect an interactive virtual manipulation selection by a particular player of said interactive, virtual, three-dimensional merchandise item game object, wherein said interactive virtual manipulation selection comprises a manipulation selection instruction to manipulate said interactive, virtual, three-dimensional merchandise item game object, said instruction received by said computer system from a particular player computer device used by said particular player to interact with said online interactive, virtual, three-dimensional session;
Respond to said interactive virtual manipulation selection with a corresponding display of a virtual presentation of said interactive, virtual, three-dimensional merchandise item game object; and
Respond to interactive virtual manipulation instructions by said particular player of said interactive, virtual, three-dimensional merchandise item game object by displaying virtual rotation of said interactive, virtual, three-dimensional merchandise item game object according to said interactive virtual manipulation instruction.

4. The computer system of claim 3, said computer system further programmed to:
Respond to an interactive keep selection by said particular player of said virtual, interactive, three-dimensional merchandise item game object with a virtual placement in an interactive, virtual, three-dimensional cart game object; and
Respond to an interactive pay selection by said particular player of said virtual, interactive, three-dimensional merchandise item game object with a charge to an account associated with said particular player.

5. The computer system of claim 1, said computer system further programmed to:
Distribute a virtual three-dimensional view of said online interactive, virtual, three-dimensional session to a plurality of player computer devices associated with a plurality of players as a massively multi-player online shopping game, said massively multi-player online shopping game interactive with said plurality of players, said massively multi-player online shopping game comprising an interactive persistent display of a plurality of virtual three-dimensional game objects.

6. The computer system of claim 1, wherein said online interactive, virtual, three-dimensional session comprises a persistent, interactive, visually traversable, visually rotatable, virtual three-dimensional online space, said computer system further programmed to:
Generate a persistent, interactive, visually traversable, visually rotatable, virtual three-dimensional representation of a plurality of interactive, virtual, three-dimensional merchandise item game objects presented in a persistent, interactive, visually traversable, virtual three-dimensional representation of a virtual store interior.

7. The computer system of claim 6, said computer system further programmed to:
Receive on a continuous basis from each player computer device of said plurality of player computer devices an indication of a player-specific virtual location within said store interior, said player-specific virtual location corresponding to a virtual location of a particular player associated with a particular player computer device of said plurality of player computer devices within said virtual store interior;
Track for each particular player, an indication of player store traversal activity by each said particular player from a particular player computer device associated with a particular computer display associated with each said particular player; and
Interact with said indication of player store traversal activity by:
displaying visual traversal of said store interior to said particular computer display; and
streaming sound to an audio speaker in communication with said particular player computer device according to said indication of player store traversal activity with respect to said player-specific virtual location within said store interior.

8. The computer system of claim 6, said computer system further programmed to:
Generate for each said particular player of said plurality of players, a player game object representation;
Receive from each player computer device of said plurality of player computer devices an indication of a player-specific virtual location of said player game object within said virtual store interior, said player-specific virtual location corresponding to a virtual location of a particular player associated with a particular player computer device of said plurality of player computer devices within said virtual store interior;
Continuously track for each particular player, a streaming indication of player store traversal activity by each player game object for each particular player from a particular player computer device associated with a particular computer display associated with said each particular player;
Continuously display to each computer display associated with each player computer device of said plurality of player computer devices a visual location within said virtual store interior of said player game object;
Continuously stream to an audio device in communication with each player computer device of said plurality of player computer devices, sound corresponding to said virtual location within said virtual store interior according to said player-specific virtual location of said player game object; and
Continuously display to each computer display associated with each player computer device of said plurality of player computer devices, each player game object associated with each particular player of said plurality of players, as tracked according to said streaming indication of player store traversal activity by each said particular player game object.

9. The computer system of claim 6, said computer system further programmed to:
Display a plurality of interactive, virtual, three-dimensional merchandise item game objects on a visually traversable, visually rotatable, virtual three-dimensional representation of a display structure;
Receive an indication of player display structure traversal activity from a particular player computer device associated with a particular computer display; and
Interact with said indication of player display structure traversal activity by displaying visual traversal of said virtual display structure to said particular computer display.

10. A computer system for dynamically creating visually rotatable, virtual three-dimensional, interactive advertising in a shared, interactive shopping session in a persistent, visually traversable, interactive, virtual three-dimensional marketplace, said computer system comprising computer software programmed to:
continuously track a location of each virtual shopping user of a plurality of virtual shopping users concurrently shopping in a virtual online marketplace;
as a particular virtual shopping user of the plurality of virtual shopping users approaches a particular virtual location within the virtual marketplace, analyze a shopping history for the particular shopping user with respect to the particular virtual location;
identify items of potential interest to the particular virtual shopping user according to analysis of said shopping history;
dynamically create an advertisement of virtual items similar to said items of interest; and
present the advertisement to a computer display associated with a computer device being used by the virtual shopping user to interact with said shared, interactive shopping session.

11. The computer system of claim 10, said computer system further comprising computer software programmed to:
dynamically create said advertisement of virtual items as a virtual, three-dimensional advertisement.

12. The computer system of claim 10, wherein said items of potential interest comprise items similar to items previously purchased or previously inspected by said particular virtual shopping user.

13. The computer system of claim 10, wherein said items of potential interest comprise items that would complement items previously purchased or previously inspected by said particular virtual shopping user.

14. A computer system for creating a proportionally scaled, persistent, interactive, visually traversable, visually rotatable, virtual three-dimensional representation of a plurality of items of merchandise within a persistent, visually traversable, virtual three-dimensional representation of a virtual store, said computer system programmed to:
receive a plurality of sets of three-dimensional display structure scan data, each set of said plurality of sets comprising three-dimensional scan data for a particular display structure of a plurality of display structures;
receive a set of defining characteristics of a virtual store;
receive a set of requirements for virtual positioning each display structure of said plurality of display structures in said virtual store;
create, according to said set of requirements, a proportionally scaled, persistent, visually traversable, virtual three-dimensional representation of said plurality of said display structures virtually positioned within a persistent, visually traversable, virtual three-dimensional representation of said virtual store for display in a computer display.

15. The computer system of claim 14, wherein said set of defining characteristics of a virtual store comprises one or more of:
store layout specifications, department specifications, isle specifications, shelving specifications, lighting specifications, platform specifications, cabinet specifications, rack specifications, display case specifications, merchandise specifications, mannequin specifications, and décor specifications.

16. The computer system of claim 15, said computer system further programmed to:
receive location data from a first shopping user regarding a virtual location within said virtual store of said first shopping user; and
generate a first virtual shopping user emoji for said first shopping user comprising a persistent, virtual three-dimensional representation of said first shopping user positioned within said virtual store at said virtual location.

17. The computer system of claim 16, said computer system further programmed to:
share a display of said first virtual shopping user emoji with respective computers associated with other shopping users virtually located within said virtual store at a given time.

18. The computer system of claim 17, said computer system further programmed to:
receive a notification by a second shopping user of a communication request for said first virtual shopping user emoji;
present a notification to the first shopping user associated with said first virtual shopping user emoji that a second shopping user requests communication; and
subject to approval by the first shopping user, facilitate two-way communication between the first shopping user and the second shopping user, said two-way communication automatically comprising foreign language cross-translation if the first shopping user and the second shopping user communicate using different primary languages, according to primary language settings for the first shopping user and primary language settings for the second shopping user.

19. A computer system that is programmed to generate instructions to activate fragrance emitting elements provided by a fragrance emitting computer device that is in communication with a shopping user computer, said fragrance emitting computer device comprising:
a wireless communication component for communicating with said shopping user computer device,
a plurality of fragrance emitting elements connected to said fragrance emitting computer device, and
a plurality of fragrances, each fragrance of said plurality of fragrances contained on a fragrance emitting element of said plurality of fragrance emitting elements;
said fragrance emitting computer device programmed to:
receive an instruction from a computer system for emitting a particular fragrance for a particular duration and with a particular intensity,
determine at least one fragrance emitting element of said plurality of fragrance emitting elements for emitting said particular fragrance, and
activate said at least one fragrance emitting element to emit a fragrance for said particular duration and with said particular intensity.

20. The computer system of claim 19, said computer system programmed to:
determine a location of a particular player object corresponding to said shopping user within a persistent, visually traversable, virtual three-dimensional representation of a virtual shopping space;
determine a fragrance instruction corresponding to said location;
generate said fragrance instruction; and
communicate said fragrance instruction to said shopping user computer device for communication to said fragrance emitting computer device.
